(19) United States
(12) Patent Application Publication
(10) Pub. No.: US 2009/0054413 A1
(43) Pub. Date: Feb. 26, 2009

(54) Novel 5,6-Dihydropyrazolo[3,4-E] [L,4]Diazepin-4 (IH) -One Derivatives for the Treatment of Asthma and Chronic Obstructive Pulmonary Disease

(75) Inventors: Krister Henriksson, Lund (SE); Annea Lisius, Lund (SE); Peter Sjo, Lund (SE); Peter Storm, Molndal (SE)

Correspondence Address: FISH & RICHARDSON P.C., P.O BOX 1022, MINNEAPOLIS, MN, 55440-1022, US

(73) Assignee: AstraZeneca AB, Sodertalje (SE)

(21) Appl. No.: 12/088,552

(22) PCT Filed: Oct. 2, 2006
(86) PCT No.: PCT/SE06/01111

§ 371 Date: Jul. 29, 2008

(30) Foreign Application Priority Data
Oct. 3, 2005 (SE) 0502176-1

(51) Int. Cl. A61K 31/551; C07D 487/04; A61P 11/06; A61P 29/00

(52) U.S. Cl. 514/221; 540/502

(57) ABSTRACT

The present invention provides a compound of a formula (I):

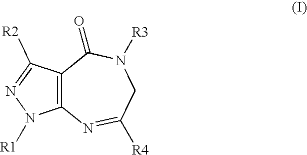

wherein the variables are defined herein; to a process for preparing such a compound; and to the use of such a compound in the treatment of a PDE 4 mediated disease state.

The present invention concerns 5,6-dihydropyrazolo[3,4-e][1,4]diazepin-4(1H)-one derivatives having pharmaceutical activity, to processes for preparing such derivatives, to pharmaceutical compositions comprising such derivatives and to the use of such derivatives as active therapeutic agents.

Phosphodiesterases (PDEs) work by converting cAMP or cGMP to AMP and GMP, or the inactive nucleotide forms incapable of activating downstream signalling pathways. The inhibition of PDEs leads to the accumulation of cAMP or cGMP, and subsequent activation of downstream pathways. PDEs comprise a large family of second messengers with 11 families and over 50 isoforms. In addition splice variants have been described for each isoform. The PDEs can be cAMP-specific (PDE4, 7, 8, 10), cGMP specific (PDE5, 6, 9) or have dual specificity (PDE1, 2, 3, 11).

cAMP is generated from ATP at the inner leaflet of the plasma membrane through the action of GPCR-regulated adenylate cyclase. Once cAMP is generated, the only way to terminate the signal is through phosphodiesterase action, degrading cAMP into 5′-AMP. Increased concentrations of cAMP are translated into cellular responses mainly by activation of cAMP-dependent protein kinase (PKA). The specific activity of PKA is in part regulated by the sub-cellular localization of PKA, which limits the phosphorylation of PKA to substrates in its near vicinity. The downstream events caused by activation of PKA appear poorly elucidated and involve many components in the initiation of signalling cascades. PDE4s have been shown to have abundant roles in regulating cell desensitisation, adaptation, signal cross-talk, cAMP compartmentalization and feedback loops, and are major regulators of cAMP homeostasis.

The physiological role implicated for elevated cAMP levels include: 1) broad suppression of the activity of many immunocompetent cells; 2) induction of airway smooth muscle relaxation; 3) suppression of smooth muscle mitogenesis; and, 4) has beneficial modulatory effects on the activity of pulmonary nerves.

PDE4 has been found to be the predominant cAMP metabolising isozyme family in immune and inflammatory cells and, along with the PDE3 family, a major contributor to cAMP metabolism in airway smooth muscle.

Over the last two decades significant attention has been devoted into the development of PDE4 selective inhibitors for the treatment of inflammatory and immune disorders including asthma, rhinitis, bronchitis, COPD, arthritis and psoriasis. A number of compounds (for example rolipram, tibenelast and denbufylline) have been reported to have impressive effects in animal models of inflammation, especially pulmonary inflammation.

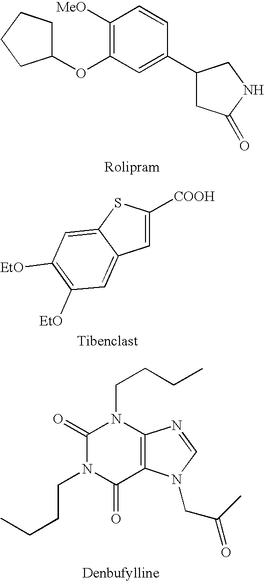

Unfortunately the clinical utility of these inhibitors has been limited by PDE4 related side-effects, including nausea, vomiting and gastric acid secretion. Recently a second generation of PDE4 inhibitors (for example cilomilast, roflumilast and AWD 12-281) has been described having significantly reduced risk of emetic side effects in animal models of emesis, thus providing the potential for an increased therapeutic ratio.

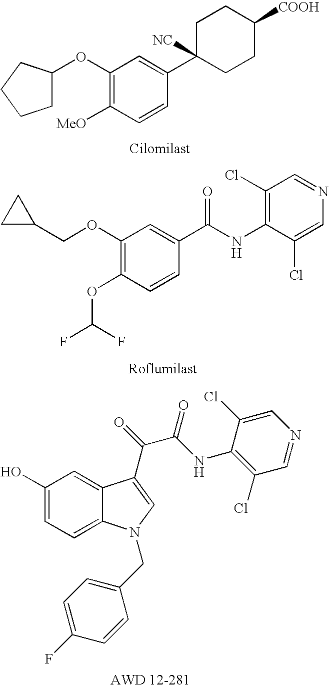

The present invention discloses novel amino pyridine sulphonamide derivatives that are inhibitors of human PDE4 and are thereby useful in therapy.

Pyrazolo[4,3-e]diazepine derivatives that are useful as PDE4 inhibitors are disclosed in WO 01/49689.

The present invention provides a compound of formula (I):

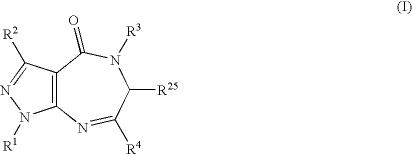

wherein:
R¹ is a 5- or 6-membered, saturated or unsaturated, carbocyclic or heterocyclic ring; when R¹ is aryl or heteroaryl then said aryl or heteroaryl is optionally substituted as recited below; when R¹ is a non-aromatic carbocycle or non-aromatic heterocyclyl then said ring is optionally substituted by C₁₋₆ alkyl;
R² is C₁₋₄ alkyl, C₁₋₄ alkoxy or C₁₋₄ alkylthio;
R³ is hydrogen or C₁₋₄ alkyl;
R⁴ is C₁₋₆ alkyl (optionally substituted by CO₂R²⁶ or C(O)NR²⁷R²⁸), optionally substituted aryl or optionally substituted heteroaryl;
R²⁵ is hydrogen or optionally substituted phenyl;
the foregoing aryl, heteroaryl and phenyl rings are, independently, optionally substituted by: halogen, cyano, nitro, hydroxy, S(O)_{q}R⁵, OC(O)NR⁶R⁷, NR⁸R⁹, NR¹⁰C(O)R¹¹, NR¹²C(O)NR¹³R¹⁴, S(O)₂NR¹⁵R¹⁶, NR¹⁷S(O)₂R¹⁸, C(O)NR¹⁹R²⁰, C(O)R²¹, CO₂R²², NR²³CO₂R²⁴, C₁₋₆ alkyl, C₁₋₆ haloalkyl, C₁₋₆ alkoxy(C₁₋₆)alkyl, C₁₋₆ alkoxy, C₁₋₆ haloalkoxy, C₁₋₆ alkoxy(C₁₋₆)alkoxy, C₁₋₆ alkylthio, O(CH₂)ₙO, phenyl(C₁₋₆)alkyl, phenoxy(C₁₋₆)alkyl or phenyl(C₁₋₆)alkoxy; the phenyl rings of the last three substituents being optionally substituted by halogen, C₁₋₄ alkyl, C₁₋₄ alkoxy, CF₃ or OCF₃;
n is 1, 2 or 3;
q is 0, 1 or 2;
R⁶ and R⁷, R¹³ and R¹⁴, R¹⁵ and R¹⁶ or R¹⁹ and R²⁰ can join to form, with the nitrogen to which they are bonded, a 5- or 6-membered ring;
R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴ and R²⁶ are, independently, C₁₋₆ alkyl (optionally substituted by halogen, hydroxy or C₁₋₆ alkoxy) or phenyl (itself optionally substituted by halogen, C₁₋₄ alkyl, C₁₋₄ alkoxy, CF₃ or OCF₃);
R⁶, R⁷, R⁸, R⁹, R¹⁰, R¹¹, R¹², R¹³, R¹⁴, R¹⁵, R¹⁶, R¹⁷, R¹⁹, R²⁰, R²¹, R²², R²³ and R²⁶ can also be hydrogen;
R²⁷ is hydrogen or C₁₋₆ alkyl;
R²⁸ is hydrogen or C₁₋₆ alkyl (optionally substituted by NH₂, NH(C₁₋₆ alkyl) or N(C₁₋₆ alkyl)₂);
or a N-oxide thereof; or a pharmaceutically acceptable salt thereof.

Certain compounds of the present invention can exist in different isomeric forms (such as enantiomers, diastereomers, geometric isomers or tautomers). The present invention covers all such isomers and mixtures thereof in all proportions.

Suitable salts include acid addition salts such as a hydrochloride, dihydrochloride, hydrobromide, phosphate, sulfate, acetate, diacetate, fumarate, maleate, malonate, succinate, tartrate, citrate, oxalate, methanesulfonate or p-toluenesulfonate.

The compounds of the invention may exist as solvates (such as hydrates) and the present invention covers all such solvates.

The compounds of the present invention are advantageous over known PDE4 inhibitors as they have a surprisingly high level of solubility (for example in water); they have low hERG channel activity; and they are selective inhibitors of PDE4 over other PDEs (that is, there is a difference of 10³ or more in the activities of the compounds of the invention against PDE4 and other PDEs).

Halogen includes fluorine, chlorine, bromine and iodine. Halogen is, for example, fluorine or chlorine.

Alkyl moieties are straight or branched chain and are, for example, methyl, ethyl, n-propyl, iso-propyl or tert-butyl. Haloalkyl is, for example C₂F₅, CF₃ or CHF₂. Alkoxy is, for example, methoxy or ethoxy; and haloalkoxy is, for example OCF₃ or OCHF₂.

Alkenyl is, for example, vinyl or prop-2-enyl. Alkynyl is, for example, propargyl.

Aryl is, for example, phenyl or naphthyl (such as phenyl).

Phenyl(C₁₋₆)alkyl is, for example, benzyl, 1-phenyleth-1-yl or 2-phenyleth-1-yl.

Phenoxy(C₁₋₆)alkyl is, for example, phenoxymethyl or 2-phenoxyeth-1-yl.

Phenyl(C₁₋₆)alkoxy is, for example, benzyloxy or 2-phenyleth-1-oxy.

The substituent O(CH₂)ₙO when on, for example, a phenyl ring provides, when n is for example 1, a methylenedioxy group.

Heteroaryl is, for example, an aromatic 5 or 6 membered ring, optionally fused to one or more other rings, comprising at least one heteroatom selected from the group comprising nitrogen, oxygen and sulphur; or an N-oxide thereof, or an S-oxide or S-dioxide thereof. Heteroaryl is, for example, furyl, thienyl (also known as thiophenyl), pyrrolyl, thiazolyl, isothiazolyl, pyrazolyl, oxazolyl, isoxazolyl, imidazolyl, [1,2,4]-triazolyl, [1,2,3]-triazolyl, pyridinyl, pyrimidinyl, pyrazinyl, pyridazinyl, indolyl, benzo[b]furyl (also known as benzfuryl), benz[b]thienyl (also known as benzthienyl or benzthiophenyl), indazolyl, benzimidazolyl, benztriazolyl, benzoxazolyl, benzthiazolyl, 1,2,3-benzothiadiazolyl, an imidazopyridinyl (such as imidazo[1,2a]pyridinyl), thieno[3,2-b]pyridin-6-yl, 1,2,3-benzoxadiazolyl (also known as benzo[1,2,3]thiadiazolyl), 1,2,3-benzothiadiazolyl, 2,1,3-benzothiadiazolyl, benzofurazan (also known as 2,1,3-benzoxadiazolyl), quinoxalinyl, a pyrazolopyridine (for example 1H-pyrazolo[3,4-b]pyridinyl), quinolinyl, isoquinolinyl or a naphthyridinyl (for example [1,6]naphthyridinyl or [1,8]naphthyridinyl); or an N-oxide thereof, or an S-oxide or S-dioxide thereof.

Heterocyclyl is, for example, a 5- or 6-membered ring comprising 1, 2 or 3 (for example 1 or 2) nitrogen, oxygen or sulphur atoms. For example heterocyclyl is pyrrolidinyl, morpholinyl, thiomorpholinyl, piperidinyl, piperazinyl, tetrahydropyranyl or tetrahydrothiopyranyl. Further examples of heterocyclyl are: tetrahydrothienyl, 1-oxidotetrahydrothienyl, 1,1-dioxidotetrahydrothienyl, 1-oxidotetrahydrothiopyranyl and 1,1-dioxidotetrahydrothiopyranyl.

A 5- or 6-membered, saturated or unsaturated, carbocyclic or heterocyclic ring is, for example, heterocyclyl, heteroaryl, phenyl, cyclopentyl or cyclohexyl.

In one particular aspect the present invention provides a compound of formula (I) wherein: R¹ is a 5- or 6-membered, saturated or unsaturated, carbocyclic or heterocyclic ring; R² is C₁₋₄ alkyl; R³ is hydrogen or C₁₋₄ alkyl; R⁴ is aryl and heteroaryl; R²⁵ is hydrogen; the aryl and heteroaryl moieties of R⁴ are, independently, optionally substituted by: halogen, cyano, nitro, hydroxy, S(O)_{q}R⁵, OC(O)NR⁶R⁷, NR⁸R⁹, NR¹⁰C(O)R¹¹, NR¹²C(O)NR¹³R¹⁴, S(O)₂NR¹⁵R¹⁶, NR¹⁷S(O)₂R¹⁸, C(O)NR¹⁹R²⁰, C(O)R²¹, CO₂R²², NR²³CO₂R²⁴, C₁₋₆ alkyl, C₁₋₆ haloalkyl, C₁₋₆ alkoxy(C₁₋₆)alkyl, C₁₋₆ alkoxy, C₁₋₆ haloalkoxy, C₁₋₆ alkoxy(C₁₋₆)alkoxy or C₁₋₆ alkylthio; q and r are, independently, 0, 1 or 2; R⁶ and R⁷, R¹³ and R¹⁴, R¹⁵ and R¹⁶, or R¹⁹ and R²⁰ can join to form, with the nitrogen to which they are bonded, a 5- or 6-membered ring; R⁵, R⁶, R⁷, R⁸, R⁹, R¹⁰, R¹¹, R¹², R¹³, R¹⁴, R¹⁵R¹⁶R¹⁷R¹⁸, R¹⁹, R²⁰, R²¹R²², R²³ and R²⁴ are, independently, hydrogen, C₁₋₆ alkyl (optionally substituted by halogen, hydroxy or C₁₋₆ alkoxy) or phenyl (itself optionally substituted by halogen, C₁₋₄ alkyl, C₁₋₄ alkoxy, CF₃ or OCF₃); R⁶, R⁷, R⁸, R⁹, R¹⁰, R¹¹, R¹², R¹³R¹⁴, R¹⁵, R¹⁶, R¹⁷, R¹⁹, R²⁰, R²¹, R²² and R²³ can also be hydrogen; or a N-oxide thereof; or a pharmaceutically acceptable salt thereof.

In a further aspect the present invention provides a compound of formula (I) wherein R¹ is cyclopentyl, or phenyl (optionally substituted as recited for aryl of R⁴ above or below).

In another aspect the present invention provides a compound of formula (I) wherein R¹ is cyclopentyl, or phenyl (optionally substituted by halo (such as fluoro), NO₂ or S(O)₂(C₁₋₄ alkyl) (such as S(O)₂CH₃)). In a further aspect R¹ is cyclopentyl.

In yet another aspect the present invention provides a compound of formula (I) wherein R² is C₁₋₄ alkyl (for example ethyl).

In a further aspect the present invention provides a compound of formula (I) wherein R³ is hydrogen.

In a still further aspect the present invention provides a compound of formula (I) wherein the aryl (such as phenyl) and heteroaryl moieties of R⁴ are, independently, optionally substituted by: halogen, cyano, nitro, S(O)_{q}R⁵, NHC(O)R¹¹, S(O)₂NR¹⁵R¹⁶, CO₂R²², C₁₋₆ alkyl, C₁₋₆ haloalkyl, C₁₋₆ alkoxy, C₁₋₆ haloalkoxy, C₃₋₁₀ cycloalkyl (itself optionally substituted by C₁₋₄ alkyl or oxo), heterocyclyl (for example morpholinyl; heterocyclyl being optionally substituted by C₁₋₄ alkyl), phenyl or heteroaryl (for example pyrazolyl, pyridinyl or pyrimidinyl); wherein any of the immediately foregoing phenyl and heteroaryl moieties are optionally substituted with halogen, S(O)ᵣ(C₁₋₄ alkyl), C₁₋₄ alkyl, CF₃ or OCF₃; wherein q and r are, independently, 0, 1 or 2; R⁵ is C₁₋₆ alkyl; R¹¹, R¹⁵, R¹⁶ and R²² are, independently, hydrogen or C₁₋₆ alkyl.

In a still further aspect the present invention provides a compound of formula (I) wherein the aryl (such as phenyl) and heteroaryl moieties of R⁴ are, independently, optionally substituted by: halogen, cyano, nitro, S(O)_{q}R⁵, NHC(O)R¹¹, S(O)₂NR¹⁵R¹⁶, CO₂R²², C₁₋₆ alkyl, C₁₋₆ haloalkyl, C₁₋₆ alkoxy or C₁₋₆ haloalkoxy; wherein q is 0, 1 or 2; R⁵ is C₁₋₆ alkyl; and R¹¹, R¹⁵, R¹⁶ and R²² are, independently, hydrogen or C₁₋₆ alkyl.

In yet another aspect the present invention provides a compound of formula (I) wherein R⁴ is phenyl optionally substituted by: halogen, cyano, nitro, S(O)_{q}R⁵, NHC(O)R¹¹, S(O)₂NR¹⁵R¹⁶, CO₂R²², C₁₋₆ alkyl, C₁₋₆ haloalkyl, C₁₋₆ alkoxy, C₁₋₆ haloalkoxy, C₃₋₁₀ cycloalkyl (itself optionally substituted by C₁₋₄ alkyl or oxo), heterocyclyl (for example morpholinyl; heterocyclyl being optionally substituted by C₁₋₄ alkyl), phenyl or heteroaryl (for example pyrazolyl, pyridinyl or pyrimidinyl); wherein any of the immediately foregoing phenyl and heteroaryl moieties are optionally substituted with halogen, S(O)ᵣ(C₁₋₄ alkyl), C₁₋₄ alkyl, CF₃ or OCF₃; wherein q and r are, independently, 0, 1 or 2; R⁵ is C₁₋₆ alkyl; R¹¹, R¹⁵, R¹⁶ and R²² are, independently, hydrogen or C₁₋₆ alkyl.

In a further aspect the present invention provides a compound of formula (I) wherein R⁴ is phenyl optionally substituted by: halogen, cyano, nitro, S(O)_{q}R⁵, NHC(O)R¹¹, S(O)₂NR¹⁵R¹⁶, CO₂R²², C₁₋₆ alkyl, C₁₋₆ haloalkyl, C₁₋₆ alkoxy or C₁₋₆ haloalkoxy; wherein q is 0, 1 or 2; R⁵ is C₁₋₆ alkyl; and R¹¹, R¹⁵, R¹⁶ and R²² are, independently, hydrogen or C₁₋₆ alkyl.

In a further aspect the present invention provides a compound of formula (I) wherein R⁴ is phenyl optionally substituted by halogen (such as fluoro, chloro or bromo), C₁₋₄ alkyl (such as methyl), C₁₋₄ alkoxy (such as methoxy), hydroxy, CO₂H, CO₂(C₁₋₄ alkyl) (such as CO₂CH₃), phenyl(C₁₋₄)alkoxy (such as OCH₂phenyl) or O(CH₂)ₙO (such as O(CH₂)₃O).

In a still further aspect the present invention provides a compound of formula (I) wherein R⁴ is phenyl optionally substituted by halogen, C₁₋₄ alkyl, CF₃, C₁₋₄ alkoxy or OCF₃.

In another aspect the present invention provides a compound of formula (I) wherein R⁴ is C₁₋₆ alkyl (such as tert-butyl).

In yet another aspect the present invention provides a compound of formula (I) wherein R²⁵ is hydrogen.

Compounds of the invention are described in the Examples. Each of the compounds of the Examples, in free base or salt form, is a further aspect of the present invention. Thus the present invention provides a compound:
1-Cyclopentyl-3-ethyl-7-phenyl-5,6-dihydropyrazolo[3,4-e][1,4]diazepin-4(1H)-one;
1-Cyclopentyl-3-ethyl-7-(4-methoxyphenyl)-5,6-dihydropyrazolo[3,4-e][1,4]diazepin-4(1H)-one;
3-Ethyl-1-(4-fluorophenyl)-7-phenyl-5,6-dihydropyrazolo[3,4-e][1,4]diazepin-4(1H)-one;
3-Ethyl-1-(3-nitrophenyl)-7-phenyl-5,6-dihydropyrazolo[3,4-e][1,4]diazepin-4(1H)-one;
3-Ethyl-1-[4-(methylsulfonyl)phenyl]-7-phenyl-5,6-dihydropyrazolo[3,4-e][1,4]diazepin-4(1H)-one,
1-Cyclopentyl-7-(3,4-dimethoxyphenyl)-3-ethyl-5,6-dihydropyrazolo[3,4-e][1,4]diazepin-4(1H)-one;
7-(4-Bromophenyl)-1-cyclopentyl-3-ethyl-5,6-dihydropyrazolo[3,4-e][1,4]diazepin-4(1H)-one;
1-Cyclopentyl-3-ethyl-7-(4-methylphenyl)-5,6-dihydropyrazolo[3,4-e][1,4]diazepin-4(1H)-one;
1-Cyclopentyl-3-ethyl-7-(2-methoxyphenyl)-5,6-dihydropyrazolo[3,4-e][1,4]diazepin-4(1H)-one;
Methyl 5-(1-cyclopentyl-3-ethyl-4-oxo-1,4,5,6-tetrahydropyrazolo[3,4-e][1,4]diazepin-7-yl)-2-hydroxybenzoate;
7-(4-Chlorophenyl)-1-cyclopentyl-3-ethyl-5,6-dihydropyrazolo[3,4-e][1,4]diazepin-4(1H)-one;
7-(4-Chlorophenyl)-1-cyclopentyl-3-ethyl-5,6-dihydropyrazolo[3,4-e][1,4]diazepin-4(1H)-one;
1-Cyclopentyl-7-(2,5-dimethoxyphenyl)-3-ethyl-5,6-dihydropyrazolo[3,4-e][1,4]diazepin-4(1H)-one;
7-[4-(Benzyloxy)phenyl]-1-cyclopentyl-3-ethyl-5,6-dihydropyrazolo[3,4-e][1,4]diazepin-4(1H)-one;
1-Cyclopentyl-7-(3,4-dihydro-2H-1,5-benzodioxepin-7-yl)-3-ethyl-5,6-dihydropyrazolo[3,4-e][1,4]diazepin-4(1H)-one;
1-Cyclopentyl-3-ethyl-7-(2-furyl)-5,6-dihydropyrazolo[3,4-e][1,4]diazepin-4(1H)-one;
7-tert-Butyl-1-cyclopentyl-3-ethyl-5,6-dihydropyrazolo[3,4-e][1,4]diazepin-4(1H)-one;
1-Cyclopentyl-3-ethyl-7-(3-methoxyphenyl)-5,6-dihydropyrazolo[3,4-e][1,4]diazepin-4(1H)-one;
Methyl 3-(1-cyclopentyl-3-ethyl-4-oxo-1,4,5,6-tetrahydropyrazolo[3,4-e][1,4]diazepin-7-yl)propanoate;
1-Cyclopentyl-3-ethyl-7-(4-fluorophenyl)-5,6-dihydropyrazolo[3,4-e][1,4]diazepin-4(1H)-one;
1-Cyclopentyl-3-ethyl-7-[4-(trifluoromethoxy)phenyl]-5,6-dihydropyrazolo[3,4-e][1,4]diazepin-4(1H)-one;
4-(1-Cyclopentyl-3-ethyl-4-oxo-1,4,5,6-tetrahydropyrazolo[3,4-e][1,4]diazepin-7-yl)benzonitrile;
1-Cyclopentyl-3-ethyl-7-[4-(methylsulfonyl)phenyl]-5,6-dihydropyrazolo[3,4-e][1,4]diazepin-4(1H)-one;
1-Cyclopentyl-3-ethyl-7-methyl-6-phenyl-5,6-dihydropyrazolo[3,4-e][1,4]diazepin-4(1H)-one;
1-Cyclopentyl-3-ethyl-5-methyl-7-phenyl-5,6-dihydropyrazolo[3,4-e][1,4]diazepin-4(1H)-one;
5-(1-Cyclopentyl-3-ethyl-4-oxo-1,4,5,6-tetrahydropyrazolo[3,4-e][1,4]diazepin-7-yl)-2-hydroxybenzoic acid;
3-(1-Cyclopentyl-3-ethyl-4-oxo-1,4,5,6-tetrahydropyrazolo[3,4-e][1,4]diazepin-7-yl)propanoic acid;
3-(1-Cyclopentyl-3-ethyl-4-oxo-1,4,5,6-tetrahydropyrazolo[3,4-e][1,4]diazepin-7-yl)-N-[2-(dimethylamino)ethyl]propanamide;
1-Cyclopentyl-3-ethyl-7-phenyl-5,6,7,8-tetrahydropyrazolo[3,4-e][1,4]diazepin-4(1H)-one;
3-(Methylthio)-1,7-diphenyl-5,6-dihydropyrazolo[3,4-e][1,4]diazepin-4(1H)-one;
1-(1,1-Dioxidotetrahydrothien-3-yl)-3-ethyl-7-phenyl-5,6-dihydropyrazolo[3,4-e][1,4]diazepin-4(1H)-one;
3-Ethyl-7-phenyl-1-tetrahydro-2H-thiopyran-4-yl-5,6-dihydropyrazolo[3,4-e][1,4]diazepin-4(1H)-one;
3-Ethyl-1-(1-oxidotetrahydro-2H-thiopyran-4-yl)-7-phenyl-5,6-dihydropyrazolo[3,4-e][1,4]diazepin-4(1H)-one;
1-(1,1-Dioxidotetrahydro-2H-thiopyran-4-yl)-3-ethyl-7-phenyl-5,6-dihydropyrazolo[3,4-e][1,4]diazepin-4(1H)-one;
1-Cyclopentyl-3-methyl-7-phenyl-5,6-dihydropyrazolo[3,4-e][1,4]diazepin-4(1H)-one; or,
3-Ethyl-1,7-diphenyl-5,6-dihydropyrazolo[3,4-e][1,4]diazepin-4(1H)-one;
   or a pharmaceutically acceptable salt thereof.

The compounds of the present invention can be prepared as described below or by adapting methods known in the art.

A compound of formula (I) can be prepared by reacting a compound of formula (III):

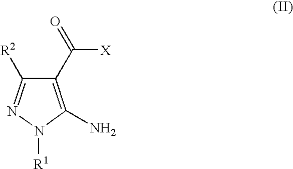

wherein X is a Cl or OH and R¹ and R² are as defined in formula (I), with an amine of formula (III):

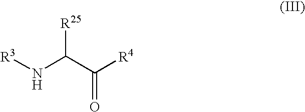

wherein R³, R⁴ and R²⁵ are as defined in formula (I), the process being carried out at a suitable temperature, generally between 25° C. and 50° C., in a suitable solvent, such as THF, in the presence of a suitable base, such as a trialkylamine for example NEt₃; and when X is OH, the process is carried out in the presence of a suitable coupling agent (such as DCC or HATU).

Compounds of formula (II) wherein X is a Cl or OH and R¹ and R² are as defined in formula (I), can be prepared by reacting a compound of formula (IV):

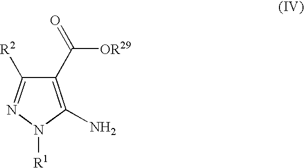

wherein R¹ and R² are as defined in formula (I), and R²⁹ is C₁₋₆ alkyl, the process being carried out at a suitable temperature, generally between 50° C. and 80° C., in a suitable mixture of solvents such as ethanol/water in the presence of a suitable base, e.g. NaOH. The resulting product, where X is OH according to formula (II), could be reacted further by treating with thionyl chloride in a suitable solvent such as dichloromethane at a suitable temperature, generally reflux temperature of the solvent, delivering compounds according to formula (II) where X is Cl. Alternatively, the resulting product, where X is OH according to formula (II), could be reacted further by treating with a suitable coupling agent such as O-(7-azabenzotriazol-1-yl)-N,N,N′,N′-tetramethyluronium hexafluorophosphate (HATU) in a suitable solvent such as dichloromethane at a suitable temperature, between 20° C. and 90° C., delivering compounds according to formula (II) where X is [1,2,3]triazolo[4,5-b]pyridin-3-ol.

Compounds of formula (IV) wherein R¹ and R² are as defined in formula (I), can be prepared by reacting a compound of formula (V):

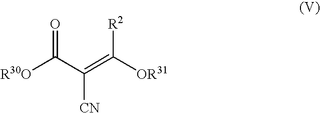

wherein R² is as defined in formula (I), and R³⁰ and R³¹ are, independently, C₁₋₆ alkyl, with a hydrazine of formula (VI):

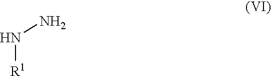

wherein R¹ is as defined in formula (I), the process being carried out at a suitable temperature, generally between 50° C. and 65° C., in a suitable solvent such as methanol in the presence of a suitable base, for example NEt₃.

Compounds of formula (I) in free or salt form can be isolated from reaction mixtures and purified in conventional manner.

Compounds of formula (III) are either commercially available or may be prepared by known methods.

Compounds of formula (V) may be prepared by the method described in Heterocyclic Chemistry, 15 (1), 2004, 67, or analogously.

Compounds of formula (VI) are either commercially available or may be prepared by known methods.

The preparations of various intermediates are described in the literature or can be prepared by routine adaptation of methods described in the literature.

In the above processes it may be desirable or necessary to protect an acid group or a hydroxy or other potentially reactive group. Suitable protecting groups and details of processes for adding and removing such groups may be found in “Protective Groups in Organic Synthesis”, 3rd Edition (1999) by Greene and Wuts.

In another aspect the present invention provides processes for the preparation of compounds of formula (I).

The invention further provides a process for the preparation of a compound of formula (Ia)

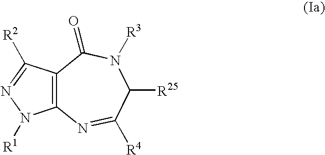

wherein:
R¹ is optionally substituted C₁₋₁₀ alkyl, or an optionally substituted 5- or 6-membered, saturated or unsaturated, carbocyclic or heterocyclic ring;
R² is optionally substituted C₁₋₁₀ alkyl, optionally substituted C₁₋₁₀ alkoxy or optionally substituted C₁₋₁₀ alkylthio;
R³ is hydrogen, optionally substituted C₁₋₁₀ alkyl or optionally substituted aryl;
R⁴ is optionally substituted C₁₋₁₀ alkyl, optionally substituted aryl or optionally substituted heteroaryl;
R²⁵ is hydrogen, optionally substituted aryl or optionally substituted heteroaryl; the process comprising reacting a compound of formula (VII):

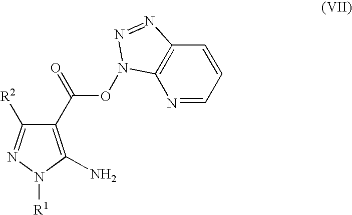

with a compound of formula (IIIa),

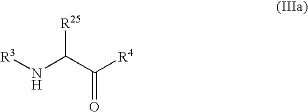

or a salt thereof, optionally in the presence of a suitable base (such as a tertiary C₁₋₆ alkylamine, for example triethylamine), in a suitable solvent (such as an ether, for example tetrahydrofuran), at a suitable temperature (for example in the range 50-150° C., such as 90-140° C.), in a sealed vessel optionally in the presence of microwaves (for example 100-200 W, such as 150 W).

A compound of formula (VII) can be prepared by reacting a compound of formula (II), wherein X is OH, with 1-hydroxy-7-azabenzotriazole (HOAT) in the presence of a suitable coupling agent (such as HATU), in the presence of a suitable base (such as a tertiary C₁₋₆ alkyl alkyl amine, for example N,N-diisopropylethylamine) in a suitable solvent (such as dichloromethane) at ambient temperature (that is 5 to 35° C.).

The advantage of the process of preparing a compound of formula (Ia) from a compound of formula (VII) is that the compound of formula (VII) (which is a reactive intermediate) can be isolated and purified thereby reducing extensive decarboxylation that can occur during workup or subsequent cyclisation in the presence of impurities or strong acids (such as hydrochloric acid). The use of microwaves further improves the yields compared to traditional heating.

In a still further aspect the present invention provides a process for preparing a compound of formula (Ia) wherein when R¹ is aryl or heteroaryl then said aryl or heteroaryl is optionally substituted as recited below; when R¹ is a non-aromatic carbocycle or heterocyclyl then said ring is optionally substituted by C₁₋₆ alkyl;

In another aspect the present invention provides a process for preparing a compound of formula (Ia) wherein the alkyl moieties, and the aryl, heteroaryl and phenyl rings of R¹, R², R³, R⁴ and R²⁵ are, independently, optionally substituted by: halogen, cyano, nitro, hydroxy, S(O)_{q}R⁵, OC(O)NR⁶R⁷, NR⁸R⁹, NR¹⁰C(O)R¹¹, NR¹²C(O)NR¹³R¹⁴, S(O)₂NR¹⁵R¹⁶, NR¹⁷S(O)₂R¹⁸, C(O)NR¹⁹R²⁰, C(O)R²¹, CO₂R²², NR²³CO₂R²⁴, C₁₋₆ alkyl, C₁₋₆ haloalkyl, C₁₋₆ alkoxy(C₁₋₆)alkyl, C₁₋₆ alkoxy, C₁₋₆ haloalkoxy, C₁₋₆ alkoxy(C₁₋₆)alkoxy, C₁₋₆ alkylthio, O(CH₂)ₙO, phenyl(C₁₋₆)alkyl, phenoxy(C₁₋₆)alkyl or phenyl(C₁₋₆)alkoxy; the phenyl rings of the last three substituents being optionally substituted by halogen, C₁₋₄ alkyl, C₁₋₄ alkoxy, CF₃ or OCF₃; n is 1, 2 or 3; q is 0, 1 or 2; R⁶ and R⁷, R¹³ and R¹⁴, R¹⁵ and R¹⁶, or R¹⁹ and R²⁰ can join to form, with the nitrogen to which they are bonded, a 5- or 6-membered ring; R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³ and R²⁴ are, independently, C₁₋₆ alkyl (optionally substituted by halogen, hydroxy or C₁₋₆ alkoxy) or phenyl (itself optionally substituted by halogen, C₁₋₄ alkyl, C₁₋₄ alkoxy, CF₃ or OCF₃); and R⁶, R⁷, R⁸, R⁹, R¹⁰, R¹¹, R¹², R¹³, R¹⁴, R¹⁵, R¹⁶, R¹⁷, R¹⁹, R²⁰, R²¹, R²² and R²³ can also be hydrogen.

In yet another aspect the present invention provides a process for preparing a compound of formula (Ia) wherein R¹, R², R³, R⁴ and R²⁵ are as defined for a compound of formula (I) above.

The compounds of formula (I) have activity as pharmaceuticals, in particular as modulators of PDE 4 receptor activity, and may be used in the treatment of inflammatory diseases, asthma or COPD.

Examples of disease states that can be treated with a compound of the invention are:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthritides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; or,
15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema.

According to a further feature of the present invention there is provided a method for treating a PDE 4 mediated disease state in a mammal, such as man, suffering from, or at risk of, said disease state, which comprises administering to a mammal in need of such treatment a therapeutically effective amount of a compound of the formula (I) or a pharmaceutically acceptable salt thereof.

The invention also provides a compound of the formula (I), or a pharmaceutically acceptable salt thereof, for use in therapy.

In another aspect the invention provides the use of a compound of formula (I), or a pharmaceutically acceptable salt thereof, in the manufacture of a medicament for use in therapy (for example modulating PDE 4 enzymatic activity).

The invention further provides the use of a compound of formula (I), or a pharmaceutically acceptable salt thereof, in the manufacture of a medicament for use in the treatment of:

1. respiratory tract: obstructive diseases of the airways including: asthma, including bronchial, allergic, intrinsic, extrinsic, exercise-induced, drug-induced (including aspirin and NSAID-induced) and dust-induced asthma, both intermittent and persistent and of all severities, and other causes of airway hyper-responsiveness; chronic obstructive pulmonary disease (COPD); bronchitis, including infectious and eosinophilic bronchitis; emphysema; bronchiectasis; cystic fibrosis; sarcoidosis; farmer's lung and related diseases; hypersensitivity pneumonitis; lung fibrosis, including cryptogenic fibrosing alveolitis, idiopathic interstitial pneumonias, fibrosis complicating anti-neoplastic therapy and chronic infection, including tuberculosis and aspergillosis and other fungal infections; complications of lung transplantation; vasculitic and thrombotic disorders of the lung vasculature, and pulmonary hypertension; antitussive activity including treatment of chronic cough associated with inflammatory and secretory conditions of the airways, and iatrogenic cough; acute and chronic rhinitis including rhinitis medicamentosa, and vasomotor rhinitis; perennial and seasonal allergic rhinitis including rhinitis nervosa (hay fever); nasal polyposis; acute viral infection including the common cold, and infection due to respiratory syncytial virus, influenza, coronavirus (including SARS) or adenovirus; or eosinophilic esophagitis;
2. bone and joints: arthritides associated with or including osteoarthritis/osteoarthrosis, both primary and secondary to, for example, congenital hip dysplasia; cervical and lumbar spondylitis, and low back and neck pain; osteoporosis; rheumatoid arthritis and Still's disease; seronegative spondyloarthropathies including ankylosing spondylitis, psoriatic arthritis, reactive arthritis and undifferentiated spondarthropathy; septic arthritis and other infection-related arthopathies and bone disorders such as tuberculosis, including Potts' disease and Poncet's syndrome; acute and chronic crystal-induced synovitis including urate gout, calcium pyrophosphate deposition disease, and calcium apatite related tendon, bursal and synovial inflammation; Behcet's disease; primary and secondary Sjogren's syndrome; systemic sclerosis and limited scleroderma; systemic lupus erythematosus, mixed connective tissue disease, and undifferentiated connective tissue disease; inflammatory myopathies including dermatomyositits and polymyositis; polymalgia rheumatica; juvenile arthritis including idiopathic inflammatory arthritides of whatever joint distribution and associated syndromes, and rheumatic fever and its systemic complications; vasculitides including giant cell arteritis, Takayasu's arteritis, Churg-Strauss syndrome, polyarteritis nodosa, microscopic polyarteritis, and vasculitides associated with viral infection, hypersensitivity reactions, cryoglobulins, and paraproteins; low back pain; Familial Mediterranean fever, Muckle-Wells syndrome, and Familial Hibernian Fever, Kikuchi disease; drug-induced arthalgias, tendonititides, and myopathies;
3. pain and connective tissue remodelling of musculoskeletal disorders due to injury [for example sports injury] or disease: arthritides (for example rheumatoid arthritis, osteoarthritis, gout or crystal arthropathy), other joint disease (such as intervertebral disc degeneration or temporomandibular joint degeneration), bone remodelling disease (such as osteoporosis, Paget's disease or osteonecrosis), polychondritits, scleroderma, mixed connective tissue disorder, spondyloarthropathies or periodontal disease (such as periodontitis);
4. skin: psoriasis, atopic dermatitis, contact dermatitis or other eczematous dermatoses, and delayed-type hypersensitivity reactions; phyto- and photodermatitis; seborrhoeic dermatitis, dermatitis herpetiformis, lichen planus, lichen sclerosus et atrophica, pyoderma gangrenosum, skin sarcoid, discoid lupus erythematosus, pemphigus, pemphigoid, epidermolysis bullosa, urticaria, angioedema, vasculitides, toxic erythemas, cutaneous eosinophilias, alopecia areata, male-pattern baldness, Sweet's syndrome, Weber-Christian syndrome, erythema multiforme; cellulitis, both infective and non-infective; panniculitis; cutaneous lymphomas, non-melanoma skin cancer and other dysplastic lesions; drug-induced disorders including fixed drug eruptions;
5. eyes: blepharitis; conjunctivitis, including perennial and vernal allergic conjunctivitis; iritis; anterior and posterior uveitis; choroiditis; autoimmune; degenerative or inflammatory disorders affecting the retina; ophthalmitis including sympathetic ophthalmitis; sarcoidosis; infections including viral, fungal, and bacterial;
6. gastrointestinal tract: glossitis, gingivitis, periodontitis; oesophagitis, including reflux; eosinophilic gastro-enteritis, mastocytosis, Crohn's disease, colitis including ulcerative colitis, proctitis, pruritis ani; coeliac disease, irritable bowel syndrome, and food-related allergies which may have effects remote from the gut (for example migraine, rhinitis or eczema);
7. abdominal: hepatitis, including autoimmune, alcoholic and viral; fibrosis and cirrhosis of the liver; cholecystitis; pancreatitis, both acute and chronic;
8. genitourinary: nephritis including interstitial and glomerulonephritis; nephrotic syndrome; cystitis including acute and chronic (interstitial) cystitis and Hunner's ulcer; acute and chronic urethritis, prostatitis, epididymitis, oophoritis and salpingitis; vulvo-vaginitis; Peyronie's disease; erectile dysfunction (both male and female);
9. allograft rejection: acute and chronic following, for example, transplantation of kidney, heart, liver, lung, bone marrow, skin or cornea or following blood transfusion; or chronic graft versus host disease;
10. CNS: Alzheimer's disease and other dementing disorders including CJD and nvCJD; amyloidosis; multiple sclerosis and other demyelinating syndromes; cerebral atherosclerosis and vasculitis; temporal arteritis; myasthenia gravis; acute and chronic pain (acute, intermittent or persistent, whether of central or peripheral origin) including visceral pain, headache, migraine, trigeminal neuralgia, atypical facial pain, joint and bone pain, pain arising from cancer and tumor invasion, neuropathic pain syndromes including diabetic, post-herpetic, and HIV-associated neuropathies; neurosarcoidosis; central and peripheral nervous system complications of malignant, infectious or autoimmune processes;
11. other auto-immune and allergic disorders including Hashimoto's thyroiditis, Graves' disease, Addison's disease, diabetes mellitus, idiopathic thrombocytopaenic purpura, eosinophilic fasciitis, hyper-IgE syndrome, antiphospholipid syndrome;
12. other disorders with an inflammatory or immunological component; including acquired immune deficiency syndrome (AIDS), leprosy, Sezary syndrome, and paraneoplastic syndromes;
13. cardiovascular: atherosclerosis, affecting the coronary and peripheral circulation; pericarditis; myocarditis, inflammatory and auto-immune cardiomyopathies including myocardial sarcoid; ischaemic reperfusion injuries; endocarditis, valvulitis, and aortitis including infective (for example syphilitic); vasculitides; disorders of the proximal and peripheral veins including phlebitis and thrombosis, including deep vein thrombosis and complications of varicose veins;
14. oncology: treatment of common cancers including prostate, breast, lung, ovarian, pancreatic, bowel and colon, stomach, skin and brain tumors and malignancies affecting the bone marrow (including the leukaemias) and lymphoproliferative systems, such as Hodgkin's and non-Hodgkin's lymphoma; including the prevention and treatment of metastatic disease and tumour recurrences, and paraneoplastic syndromes; or,
15. gastrointestinal tract: Coeliac disease, proctitis, eosinopilic gastro-enteritis, mastocytosis, Crohn's disease, ulcerative colitis, microscopic colitis, indeterminant colitis, irritable bowel disorder, irritable bowel syndrome, non-inflammatory diarrhea, food-related allergies which have effects remote from the gut, e.g., migraine, rhinitis and eczema;
in a mammal (for example man).

In a further aspect the invention provides a compound of formula (I), or a pharmaceutically acceptable salt thereof, for use in the treatment of asthma {such as bronchial, allergic, intrinsic, extrinsic or dust asthma, particularly chronic or inveterate asthma (for example late asthma or airways hyper-responsiveness)}; or COPD.

In a still further aspect a compound of formula (I), or a pharmaceutically acceptable salt thereof, is useful in the treatment of COPD.

The present invention also provides the use of a compound of formula (I), or a pharmaceutically acceptable salt thereof, in the manufacture of a medicament for use in the treatment of asthma {such as bronchial, allergic, intrinsic, extrinsic or dust asthma, particularly chronic or inveterate asthma (for example late asthma or airways hyper-responsiveness)}; or COPD.

In order to use a compound of the invention, or a pharmaceutically acceptable salt thereof, for the therapeutic treatment of a mammal, such as man, said ingredient is normally formulated in accordance with standard pharmaceutical practice as a pharmaceutical composition. Therefore in another aspect the present invention provides a pharmaceutical composition which comprises a compound of the formula (I), or a pharmaceutically acceptable salt thereof (active ingredient), and a pharmaceutically acceptable adjuvant, diluent or carrier.

In a further aspect the present invention provides a process for the preparation of said composition which comprises mixing active ingredient with a pharmaceutically acceptable adjuvant, diluent or carrier. Depending on the mode of administration, the pharmaceutical composition will, for example, comprise from 0.05 to 99% w (percent by weight), such as from 0.05 to 80% w, for example from 0.10 to 70% w, such as from 0.10 to 50% w, of active ingredient, all percentages by weight being based on total composition.

The pharmaceutical compositions of this invention may be administered in standard manner for the disease condition that it is desired to treat, for example by topical (such as to the lung and/or airways or to the skin), inhalation, oral, rectal or parenteral administration. For these purposes the compounds of this invention may be formulated by means known in the art. A suitable pharmaceutical composition of this invention is one suitable for oral administration in unit dosage form, for example a tablet or capsule which contains between 0.1 mg and 1 g of active ingredient.

Each patient may receive, for example, a dose of 0.001 mgkg⁻¹ to 100 mgkg⁻¹, for example in the range of 0.1 mgkg⁻¹ to 20 mgkg⁻¹, of the active ingredient administered, for example, 1 to 4 times per day.

The invention further relates to a combination therapy wherein a compound of the invention, or a pharmaceutically acceptable salt thereof, or a pharmaceutical composition or formulation comprising a compound of the invention, is administered concurrently or sequentially or as a combined preparation with another therapeutic agent or agents, for the treatment of one or more of the conditions listed.

In particular, for the treatment of the inflammatory diseases such as (but not restricted to) rheumatoid arthritis, osteoarthritis, asthma, allergic rhinitis, chronic obstructive pulmonary disease (COPD), psoriasis, and inflammatory bowel disease, the compounds of the invention may be combined with agents listed below.

Non-steroidal anti-inflammatory agents (hereinafter NSAIDs) including non-selective cyclo-oxygenase COX-1/COX-2 inhibitors whether applied topically or systemically (such as piroxicam, diclofenac, propionic acids such as naproxen, flurbiprofen, fenoprofen, ketoprofen and ibuprofen, fenamates such as mefenamic acid, indomethacin, sulindac, azapropazone, pyrazolones such as phenylbutazone, salicylates such as aspirin); selective COX-2 inhibitors (such as meloxicam, celecoxib, rofecoxib, valdecoxib, lumarocoxib, parecoxib and etoricoxib); cyclo-oxygenase inhibiting nitric oxide donors (CINODs); glucocorticosteroids (whether administered by topical, oral, intramuscular, intravenous, or intra-articular routes); methotrexate; leflunomide; hydroxychloroquine; d-penicillamine; auranofin or other parenteral or oral gold preparations; analgesics; diacerein; intra-articular therapies such as hyaluronic acid derivatives; and nutritional supplements such as glucosamine.

The present invention still further relates to the combination of a compound of the invention, or a pharmaceutically acceptable salt thereof, together with a cytokine or agonist or antagonist of cytokine function, (including agents which act on cytokine signalling pathways such as modulators of the SOCS system) including alpha-, beta-, and gamma-interferons; insulin-like growth factor type I (IGF-1); interleukins (IL) including IL1 to 17, and interleukin antagonists or inhibitors such as anakinra; tumour necrosis factor alpha (TNF-α) inhibitors such as anti-TNF monoclonal antibodies (for example infliximab; adalimumab, and CDP-870) and TNF receptor antagonists including immunoglobulin molecules (such as etanercept) and low-molecular-weight agents such as pentoxyfylline.

In addition the invention relates to a combination of a compound of the invention, or a pharmaceutically acceptable salt thereof, with a monoclonal antibody targeting B-Lymphocytes (such as CD20 (rituximab), MRA-aIL16R and T-Lymphocytes, CTLA4-Ig, HuMax Il-15).

The present invention still further relates to the combination of a compound of the invention, or a pharmaceutically acceptable salt thereof, with a modulator of chemokine receptor function such as an antagonist of CCR1, CCR2, CCR2A, CCR2B, CCR3, CCR4, CCR5, CCR6, CCR7, CCR8, CCR9, CCR10 and CCR11 (for the C-C family); CXCR1, CXCR2, CXCR3, CXCR4 and CXCR5 (for the C-X-C family) and CX₃CR1 for the C-X₃-C family.

The present invention further relates to the combination of a compound of the invention, or a pharmaceutically acceptable salt thereof, with an inhibitor of matrix metalloprotease (MMPs), i.e., the stromelysins, the collagenases, and the gelatinases, as well as aggrecanase; for example collagenase-1 (MMP-1), collagenase-2 (MMP-8), collagenase-3 (MMP-13), stromelysin-1 (MMP-3), stromelysin-2 (MMP-10), and stromelysin-3 (MMP-11) and MMP-9 and MMP-12, including agents such as doxycycline.

The present invention still further relates to the combination of a compound of the invention, or a pharmaceutically acceptable salt thereof, and a leukotriene biosynthesis inhibitor, 5-lipoxygenase (5-LO) inhibitor or 5-lipoxygenase activating protein (FLAP) antagonist such as; zileuton; ABT-761; fenleuton; tepoxalin; Abbott-79175; Abbott-85761; a N-(5-substituted)-thiophene-2-alkylsulfonamide; 2,6-di-tert-butylphenolhydrazones; a methoxytetrahydropyrans such as Zeneca ZD-2138; the compound SB-210661; a pyridinyl-substituted 2-cyanonaphthalene compound such as L-739,010; a 2-cyanoquinoline compound such as L-746,530; or an indole or quinoline compound such as MK-591, MK-886, and BAY x 1005.

The present invention further relates to the combination of a compound of the invention, or a pharmaceutically acceptable salt thereof, and a receptor antagonist for leukotrienes (LT) B4, LTC4, LTD4, and LTE4. selected from the group consisting of the phenothiazin-3-yls such as L-651,392; amidino compounds such as CGS-25019c; benzoxalamines such as ontazolast; benzenecarboximidamides such as BIIL 284/260; and compounds such as zafirlukast, ablukast, montelukast, pranlukast, verlukast (MK-679), RG-12525, Ro-245913, iralukast (CGP 45715A), and BAY x 7195.

The present invention still further relates to the combination of a compound of the invention, or a pharmaceutically acceptable salt thereof, and a phosphodiesterase (PDE) inhibitor such as a methylxanthanine including theophylline and aminophylline; a selective PDE isoenzyme inhibitor including another PDE4 inhibitor or an inhibitor of the isoform PDE4D, or an inhibitor of PDE5.

The present invention further relates to the combination of a compound of the invention, or a pharmaceutically acceptable salt thereof, and a histamine type 1 receptor antagonist such as cetirizine, levocetirizine, loratadine, desloratadine, fexofenadine, acrivastine, terfenadine, astemizole, azelastine, levocabastine, chlorpheniramine, promethazine, cyclizine, or mizolastine; applied orally, topically or parenterally.

The present invention still further relates to the combination of a compound of the invention, or a pharmaceutically acceptable salt thereof, and a proton pump inhibitor (such as omeprazole) or a gastroprotective histamine type 2 receptor antagonist.

The present invention flirter relates to the combination of a compound of the invention, or a pharmaceutically acceptable salt thereof, and an antagonist of the histamine type 4 receptor.

The present invention still further relates to the combination of a compound of the invention, or a pharmaceutically acceptable salt thereof, and an alpha-1/alpha-2 adrenoceptor agonist vasoconstrictor sympathomimetic agent, such as propylhexedrine, phenylephrine, phenylpropanolamine, ephedrine, pseudoephedrine, naphazoline hydrochloride, oxymetazoline hydrochloride, tetrahydrozoline hydrochloride, xylometazoline hydrochloride, tramazoline hydrochloride or ethylnorepinephrine hydrochloride.

The present invention further relates to the combination of a compound of the invention, or a pharmaceutically acceptable salt thereof, and an anticholinergic agent including muscarinic receptor (M1, M2, and M3) antagonist such as atropine, hyoscine, glycopyrrrolate, ipratropium bromide, tiotropium bromide, oxitropium bromide, pirenzepine or telenzepine.

The present invention still further relates to the combination of a compound of the invention, or a pharmaceutically acceptable salt thereof, and a beta-adrenoceptor agonist (including beta receptor subtypes 1-4) such as isoprenaline, salbutamol, formoterol, salmeterol, terbutaline, orciprenaline, bitolterol mesylate, or pirbuterol, or a chiral enantiomer thereof.

The present invention further relates to the combination of a compound of the invention, or a pharmaceutically acceptable salt thereof, and a chromone, such as sodium cromoglycate or nedocromil sodium.

The present invention still further relates to the combination of a compound of the invention, or a pharmaceutically acceptable salt thereof, with a glucocorticoid, such as flunisolide, triamcinolone acetonide, beclomethasone dipropionate, budesonide, fluticasone propionate, ciclesonide or mometasone furoate.

The present invention further relates to the combination of a compound of the invention, or a pharmaceutically acceptable salt thereof, with an agent that modulates a nuclear hormone receptor such as PPARs.

The present invention still further relates to the combination of a compound of the invention, or a pharmaceutically acceptable salt thereof, together with an immunoglobulin (Ig) or Ig preparation or an antagonist or antibody modulating Ig function such as anti-IgE (for example omalizumab).

The present invention further relates to the combination of a compound of the invention, or a pharmaceutically acceptable salt thereof, and another systemic or topically-applied anti-inflammatory agent, such as thalidomide or a derivative thereof, a retinoid, dithranol or calcipotriol.

The present invention still further relates to the combination of a compound of the invention, or a pharmaceutically acceptable salt thereof, and combinations of aminosalicylates and sulfapyridine such as sulfasalazine, mesalazine, balsalazide, and olsalazine; and immunomodulatory agents such as the thiopurines, and corticosteroids such as budesonide.

The present invention further relates to the combination of a compound of the invention, or a pharmaceutically acceptable salt thereof, together with an antibacterial agent such as a penicillin derivative, a tetracycline, a macrolide, a beta-lactam, a fluoroquinolone, metronidazole, an inhaled aminoglycoside; an antiviral agent including acyclovir, famciclovir, valaciclovir, ganciclovir, cidofovir, amantadine, rimantadine, ribavirin, zanamavir and oseltamavir; a protease inhibitor such as indinavir, nelfinavir, ritonavir, and saquinavir; a nucleoside reverse transcriptase inhibitor such as didanosine, lamivudine, stavudine, zalcitabine or zidovudine; or a non-nucleoside reverse transcriptase inhibitor such as nevirapine or efavirenz.

The present invention still further relates to the combination of a compound of the invention, or a pharmaceutically acceptable salt thereof, and a cardiovascular agent such as a calcium channel blocker, a beta-adrenoceptor blocker, an angiotensin-converting enzyme (ACE) inhibitor, an angiotensin-2 receptor antagonist; a lipid lowering agent such as a statin or a fibrate; a modulator of blood cell morphology such as pentoxyfylline; thrombolytic, or an anticoagulant such as a platelet aggregation inhibitor.

The present invention further relates to the combination of a compound of the invention, or a pharmaceutically acceptable salt thereof, and a CNS agent such as an antidepressant (such as sertraline), an anti-Parkinsonian drug (such as deprenyl, L-dopa, ropinirole, pramipexole, a MAOB inhibitor such as selegine and rasagiline, a comP inhibitor such as tasmar, an A-2 inhibitor, a dopamine reuptake inhibitor, an NMDA antagonist, a nicotine agonist, a dopamine agonist or an inhibitor of neuronal nitric oxide synthase), or an anti-Alzheimer's drug such as donepezil, rivastigmine, tacrine, a COX-2 inhibitor, propentofylline or metrifonate.

The present invention still further relates to the combination of a compound of the invention, or a pharmaceutically acceptable salt thereof, and an agent for the treatment of acute or chronic pain, such as a centrally or peripherally-acting analgesic (for example an opioid or derivative thereof), carbamazepine, phenyloin, sodium valproate, amitryptiline or other anti-depressant agents, paracetamol, or a non-steroidal anti-inflammatory agent.

The present invention further relates to the combination of a compound of the invention, or a pharmaceutically acceptable salt thereof, together with a parenterally or topically-applied (including inhaled) local anaesthetic agent such as lignocaine or a derivative thereof.

A compound of the present invention, or a pharmaceutically acceptable salt thereof, can also be used in combination with an anti-osteoporosis agent including a hormonal agent such as raloxifene, or a biphosphonate such as alendronate.

The present invention still further relates to the combination of a compound of the invention, or a pharmaceutically acceptable salt thereof, together with a: (i) tryptase inhibitor; (ii) platelet activating factor (PAF) antagonist; (iii) interleukin converting enzyme (ICE) inhibitor; (iv) IMPDH inhibitor; (v) adhesion molecule inhibitors including VLA-4 antagonist; (vi) cathepsin; (vii) kinase inhibitor such as an inhibitor of tyrosine kinase (such as Btk, Itk, Jak3 or MAP, for example Gefitinib or Imatinib mesylate), a serine/threonine kinase (such as an inhibitor of a MAP kinase such as p38, JNK, protein kinase A, B or C, or IKK), or a kinase involved in cell cycle regulation (such as a cylin dependent kinase); (viii) glucose-6 phosphate dehydrogenase inhibitor; (ix) kinin-B.sub1.- or B.sub2.-receptor antagonist; (x) anti-gout agent, for example colchicine; (xi) xanthine oxidase inhibitor, for example allopurinol; (xii) uricosuric agent, for example probenecid, sulfinpyrazone or benzbromarone; (xiii) growth hormone secretagogue; (xiv) transforming growth factor (TGFβ); (xv) platelet-derived growth factor (PDGF); (xvi) fibroblast growth factor for example basic fibroblast growth factor (bFGF); (xvii) granulocyte macrophage colony stimulating factor (GM-CSF); (xviii) capsaicin cream; (xix) tachykinin NK.sub1. or NK.sub3. receptor antagonist such as NKP-608C, SB-233412 (talnetant) or D-4418; (xx) elastase inhibitor such as UT-77 or ZD-0892; (xxi) TNF-alpha converting enzyme inhibitor (TACE); (xxii) induced nitric oxide synthase (iNOS) inhibitor; (xxiii) chemoattractant receptor-homologous molecule expressed on TH2 cells, (such as a CRTH2 antagonist); (xxiv) inhibitor of p38; (xxv) agent modulating the function of Toll-like receptors (TLR), (xxvi) agent modulating the activity of purinergic receptors such as P2X7; (xxvii) inhibitor of transcription factor activation such as NFkB, API, or STATS; or (xxviii) a non-steroidal glucocorticoid receptor agonist.

Where such a combination is to be administered by inhalation, then the one or more agents in addition to a compound of formula (I), or a pharmaceutically acceptable salt thereof, can be selected from the list comprising:
a β2 adrenoceptor agonist such as metaproterenol, isoproterenol, isoprenaline, albuterol, salbutamol, formoterol, salmeterol, terbutaline, orciprenaline, bitolterol mesylate, pirbuterol or indacaterol;
a glucocorticoid receptor agonist, {for example a non-steroidal glucocorticoid receptor agonist, or steroidal glucocorticoid receptor agonist (such as budesonide)};
a muscarinic receptor antagonist (for example a M1, M2 or M3 antagonist, such as a selective M3 antagonist) such as ipratropium bromide, tiotropium bromide, oxitropium bromide, pirenzepine, telenzepine or a glycopyrromium bromide (such as R,R-glycopyrronium bromide or a mixture of R,S- and S,R-glycopyrronium bromide);
a modulator of chemokine receptor function (such as a CCR1 receptor antagonist); or,
an inhibitor of p38 kinase function.

A compound of the invention, or a pharmaceutically acceptable salt thereof, can also be used in combination with an existing therapeutic agent for the treatment of cancer, for example suitable agents include:

(i) an antiproliferative/antineoplastic drug or a combination thereof, as used in medical oncology, such as an alkylating agent (for example cis-platin, carboplatin, cyclophosphamide, nitrogen mustard, melphalan, chlorambucil, busulphan or a nitrosourea); an antimetabolite (for example an antifolate such as a fluoropyrimidine like 5-fluorouracil or tegafur, raltitrexed, methotrexate, cytosine arabinoside, hydroxyurea, gemcitabine or paclitaxel); an antitumour antibiotic (for example an anthracycline such as adriamycin, bleomycin, doxorubicin, daunomycin, epirubicin, idarubicin, mitomycin-C, dactinomycin or mithramycin); an antimitotic agent (for example a vinca alkaloid such as vincristine, vinblastine, vindesine or vinorelbine, or a taxoid such as taxol or taxotere); or a topoisomerase inhibitor (for example an epipodophyllotoxin such as etoposide, teniposide, amsacrine, topotecan or a camptothecin);
(ii) a cytostatic agent such as an antioestrogen (for example tamoxifen, toremifene, raloxifene, droloxifene or iodoxyfene), an oestrogen receptor down regulator (for example fulvestrant), an antiandrogen (for example bicalutamide, flutamide, nilutamide or cyproterone acetate), a LHRH antagonist or LHRH agonist (for example goserelin, leuprorelin or buserelin), a progestogen (for example megestrol acetate), an aromatase inhibitor (for example as anastrozole, letrozole, vorazole or exemestane) or an inhibitor of 5α-reductase such as finasteride;
(iii) an agent which inhibits cancer cell invasion (for example a metalloproteinase inhibitor like marimastat or an inhibitor of urokinase plasminogen activator receptor function);
(iv) an inhibitor of growth factor function, for example: a growth factor antibody (for example the anti-erb b2 antibody trastuzumab, or the anti-erb b1 antibody cetuximab [C225]), a farnesyl transferase inhibitor, a tyrosine kinase inhibitor or a serine/threonine kinase inhibitor, an inhibitor of the epidermal growth factor family (for example an EGFR family tyrosine kinase inhibitor such as N-(3-chloro-4-fluorophenyl)-7-methoxy-6-(3-morpholinopropoxy)quinazolin-4-amine (gefitinib, AZD1839), N-(3-ethynylphenyl)-6,7-bis(2-methoxyethoxy)quinazolin-4-amine (erlotinib, OSI-774) or 6-acrylamido-N-(3-chloro-4-fluorophenyl)-7-(3-morpholinopropoxy)quinazolin-4-amine (CI 1033)), an inhibitor of the platelet-derived growth factor family, or an inhibitor of the hepatocyte growth factor family;
(v) an antiangiogenic agent such as one which inhibits the effects of vascular endothelial growth factor (for example the anti-vascular endothelial cell growth factor antibody bevacizumab, a compound disclosed in WO 97/22596, WO 97/30035, WO 97/32856 or WO 98/13354), or a compound that works by another mechanism (for example linomide, an inhibitor of integrin αv,β3 function or an angiostatin);
(vi) a vascular damaging agent such as combretastatin A4, or a compound disclosed in WO 99/02166, WO 00/40529, WO 00/41669, WO 01/92224, WO 02/04434 or WO 02/08213;
(vii) an agent used in antisense therapy, for example one directed to one of the targets listed above, such as ISIS 2503, an anti-ras antisense;
(viii) an agent used in a gene therapy approach, for example approaches to replace aberrant genes such as aberrant p53 or aberrant BRCA1 or BRCA2, GDEPT (gene-directed enzyme pro-drug therapy) approaches such as those using cytosine deaminase, thymidine kinase or a bacterial nitroreductase enzyme and approaches to increase patient tolerance to chemotherapy or radiotherapy such as multi-drug resistance gene therapy; or,
(ix) an agent used in an immunotherapeutic approach, for example ex-vivo and in-vivo approaches to increase the immunogenicity of patient tumour cells, such as transfection with cytokines such as interleukin 2, interleukin 4 or granulocyte-macrophage colony stimulating factor, approaches to decrease T-cell anergy, approaches using transfected immune cells such as cytokine-transfected dendritic cells, approaches using cytokine-transfected tumour cell lines and approaches using anti-idiotypic antibodies.

The invention will now be illustrated by the following Examples.

¹H NMR and ¹³C NMR spectra were recorded on a Varian Inova 400 MHz or a Varian Mercury-VX 300 MHz instrument. The central peaks of chloroform-d (δ_{H} 7.27 ppm), dimethylsulfoxide-d₆ (δ_{H} 2.50 ppm), acetonitrile-d₃ (δ_{H} 1.95 ppm) or methanol-d₄ (δ_{H} 3.31 ppm) were used as internal references. Column chromatography was carried out using silica gel (0.040-0.063 mm, Merck). Unless stated otherwise, starting materials were commercially available or readily prepared by standard methods from known starting materials. All solvents and commercial reagents were of laboratory grade and were used as received. In Examples where microwaves are used the process is carried out in a sealed vessel.

The following method was used for LC/MS analysis:

Instrument Agilent 1100; Column Waters Symmetry 2.1×30 mm; Mass APCI; Flow rate 0.7 mL/min; Wavelength 254 nm; Solvent A: water+0.1% TFA; Solvent B: acetonitrile+0.1% TFA; Gradient 15-95%/B 2.7 min, 95% B 0.3 min.

The following methods were used for LC analysis:

Method A: Instrument Agilent 1100; Column: Kromasil C18 100×3 mm, 5μ particle size, Solvent A: 0.1% TFA/water, Solvent B: 0.08% TFA/acetonitrile Flow: 1 mL/min, Gradient 10-100%/B 20 min, 100% B1 min. Absorption was measured at 220, 254 and 280 nm.

Method B: Instrument Agilent 1100; Column: XTerra C 8, 100×3 mm, 5μ particle size, Solvent A: 15 mM NH3/water, Solvent B: acetonitrile Flow: 1 mL/min, Gradient 10-100%/B 20 min, 100% B 1 min. Absorption was measured at 220, 254 and 280 nm.

The following abbreviations are used:

HATU O-(7-Azabenzotriazol-1-yl)-N,N,N′,N′-tetramethyluronium hexafluorophosphate;
HOAT 1-Hydroxy-7-azabenzotriazole;

### DIEA N,N-Diisopropylethylamine;

### THF Tetrahydrofuran;

h hour
NMP 1-N-Methyl-2-pyrrolidinone.

### EXAMPLE 1

This Example illustrates the preparation of 1-Cyclopentyl-3-ethyl-7-phenyl-5,6-dihydropyrazolo[3,4-e][1,4]diazepin-4(1H)-one.

### a) Ethyl 1-cyclopentyl-3-ethyl-5-methyl-1H-pyrazole-4-carboxylate

Ethyl 2-cyano-3-ethoxypent-2-enoate (5 g, 25.3 mmol), cyclopentylhydrazine hydrochloride (4.16 g, 30.4 mmol) and triethylamine (17.7 ml, 0.128 mol) were dissolved in methanol (100 mL) and refluxed for 16 h. The solvent was removed under vacuum, after which the residue was redissolved in dichloromethane and washed with water. The organic solvent was dried over Na₂SO₄, filtered and removed in vacuum. The residue was purified by flash chromatography (SiO₂, heptane/EtOAc 1/1) to give the title compound (4.92 g, 77%).

¹H NMR (CDCl₃): δ 5.09 (2H, br s); 4.28 (2H, q); 4.29 (1H, pent); 2.76 (2H, q); 2.15-1.85 (6H, m); 1.73-1.60 (2H, m); 1.35 (3H, t); 1.21 (3H, t).

APCI-MS m/z: 252 [MH⁺].

### b) 5-Amino-1-cyclopentyl-3-ethyl-1H-pyrazole-4-carboxylic acid

Ethyl 1-cyclopentyl-3-ethyl-5-methyl-1H-pyrazole-4-carboxylate (0.2 g, 0.8 mmol) was dissolved in ethanol (95%, 2 mL) and 3.5 M NaOH (aq) was added (0.8 mL). The solution was refluxed for 18 h. The reaction mixture was cooled to room temperature and carefully acidified (AcOH). Ethyl acetate and water was added, the organic solvent separated and the water was extracted twice with ethyl acetate. The organic solvent was dried over Na₂SO₄, filtered and removed in vacuum. The crude product was used directly.

APCI-MS m/z: 224 [MH⁺].

### c) 1-Cyclopentyl-3-ethyl-7-phenyl-5,6-dihydropyrazolo[3,4-e][1,4]diazepin-4(1H)-one

The pyrazole 5-amino-1-cyclopentyl-3-ethyl-1H-pyrazole-4-carboxylic acid (0.21 g, 0.96 mmol) was dissolved in dichloromethane (5 mL), and SOCl₂ (1 mL) was added. The mixture was refluxed for 1 h, after which the excess reagent was removed under vacuum. The solid residue was dissolved in THF (5 mL), 2-amino-1-phenylethanone hydrochloride (1.38 g, 8 mmol) and triethylamine (1.15 mL, 15.7 mmol) was added and the mixture stirred at room temperature for 24 h. The solvent was removed under vacuum, the residue redissolved in ethyl acetate and washed twice with 0.5 M aqueous citric acid. The organic solvent was dried over Na₂SO₄, filtered and removed in vacuum. The residue was purified by preparative HPLC to give the title compound (27 mg, 8%).

¹H NMR (DMSO-d₆): δ 8.13-8.09 (2H, m); 7.69 (1H, t); 7.62-7.53 (3H, m); 5.09 (1H, pent); 4.08 (2H, d); 2.82 (2H, q); 2.10-1.80 (6H, m) 1.72-1.59; (2H, m); 1.20 (3H, t).

APCI-MS m/z: 323 [MH⁺].

LC (method A) rt=11.4 min

### EXAMPLE 2

This Example illustrates the preparation of 1-cyclopentyl-3-ethyl-7-(4-methoxyphenyl)-5,6-dihydropyrazolo[3,4-e][1,4]diazepin-4(1H)-one

The title compound was synthesised in an analogous method to Example 1 by reacting 5-amino-1-cyclopentyl-3-ethyl-1H-pyrazole-4-carboxylic acid (0.19 g, 0.84 mmol) with 2-amino-1-(4-methoxyphenyl)ethanone hydrochloride (1.71 g, 8.5 mmol). Following purification by preparative HPLC, the title compound was isolated (15 mg, 5%).

¹H NMR (DMSO-d₆): δ 8.09 (2H, d); 7.63 (1H, t); 7.11 (2H, d); 5.07 (1H, pent); 4.05 (2H, d); 3.86 (3H, s); 2.81 (2H, q); 2.07-1.80 (6H, m) 1.70-1.59; (2H, m); 1.19 (3H, t).

APCI-MS m/z: 353 [MH⁺].

LC (method A) rt=11.4 min

### EXAMPLE 3

This Example illustrates the preparation of 3-ethyl-1-(4-fluorophenyl)-7-phenyl-5,6-dihydropyrazolo[3,4-e][1,4]diazepin-4(1H)-one.

### a) Ethyl 5-amino-3-ethyl-1-(4-fluorophenyl)-1H-pyrazole-4-carboxylate

Ethyl 2-cyano-3-ethoxypent-2-enoate (1.02 g, 5.2 mmol), 4-fluorophenylhydrazine hydrochloride (0.99 g, 6.1 mmol) and triethylamine (3.55 ml, 25.5 mmol) were dissolved in methanol (20 mL) and refluxed for 16 h. The solvent was removed under vacuum, after which the residue was redissolved in dichloromethane and washed with water. The organic solvent was dried over Na₂SO₄, filtered and removed in vacuum. The residue was purified by flash chromatography (SiO₂, heptane/EtOAc 1/1) to give the title compound (0.49 g, 34%).

¹H NMR (CDCl₃): δ 7.56-7.50 (2H, m); 7.24-7.18 (2H, m); 5.38 (2H, br s); 4.34 (2H, q); 2.87 (2H, q); 1.39 (3H, t); 1.30 (3H, t).

APCI-MS m/z: 278 [MH⁺].

LC (method A) rt=10.3 min

### b) 5-Amino-3-ethyl-1-(4-fluorophenyl)-1H-pyrazole-4-carboxylic acid

Ethyl 5-amino-3-ethyl-1-(4-fluorophenyl)-1H-pyrazole-4-carboxylate (0.46 g, 1.7 mmol) was dissolved in ethanol (95%, 10 mL) and 3.5 M NaOH (aq) was added (1.6 mL). The solution was refluxed for 18 h. The reaction mixture was cooled to room temperature and carefully acidified (AcOH). Ethyl acetate and water was added, the organic solvent separated and the water was extracted twice with ethyl acetate. The organic solvent was extracted with 1M NaHCO₃ (aq) twice. The basic water phase was acidified with acetic acid and extracted twice with ethyl acetate. The organic solvent was dried over Na₂SO₄, filtered and removed in vacuum, delivering pure title compound (0.35 g, 85%).

¹H NMR (DMSO-d₆): δ 7.55 (2H, dd); 7.34 (2H, dd); 6.27 (2H, br s); 2.68 (2H, q); 1.16 (3H, t).

APCI-MS m/z: 250 [MH⁺].

LC (method A) rt=6.2 min

### c) 3-Ethyl-1-(4-fluorophenyl)-7-phenyl-5,6-dihydropyrazolo[3,4-e][1,4]diazepin-4(1H)-one

The pyrazole 5-amino-3-ethyl-1-(4-fluorophenyl)-1H-pyrazole-4-carboxylic acid (0.15 g, 0.60 mmol) was dissolved in dichloromethane (5 mL), and SOCl₂ (1 mL) was added. The mixture was refluxed for 1 h, after which the excess reagent was removed under vacuum. The solid residue was dissolved in THF (5 mL), 2-amino-1-phenylethanone hydrochloride (1.00 g, 5.9 mmol) and triethylamine (0.84 mL, 6 mmol) was added and the mixture stirred at 40° C. for 19 h. The solvent was removed under vacuum, the residue redissolved in ethyl acetate and washed twice with 0.5 M aqueous citric acid. The organic solvent was dried over Na₂SO₄, filtered and removed in vacuum. The residue was purified by preparative HPLC to give the title compound (100 mg, 48%).

¹H NMR (DMSO-d₆): δ 8.02 (2H, d); 7.92 (1H, t); 7.74 (2H, dd); 7.63-7.52 (3H, m); 7.39 (2H, t); 4.22 (2H, d); 2.93 (2H, q); 1.27 (3H, t).

APCI-MS m/z: 349 [MH⁺].

LC (method A) rt=10.8 min

### EXAMPLE 4

This Example illustrates the preparation of 3-ethyl-1-(3-nitrophenyl)-7-phenyl-5,6-dihydropyrazolo[3,4-e][1,4]diazepin-4(1H)-one

### a) Ethyl 5-amino-3-ethyl-1-(3-nitrophenyl)-1H-pyrazole-4-carboxylate

Ethyl 2-cyano-3-ethoxypent-2-enoate (0.52 g, 2.64 mmol), (3-nitrophenyl)hydrazine hydrochloride (0.59 g, 3.1 mmol) and triethylamine (1.8 ml, 12.9 mmol) were dissolved in methanol (10 mL) and refluxed for 16 h. The solvent was removed under vacuum, after which the residue was redissolved in dichloromethane and washed with water. The organic solvent was dried over Na₂SO₄, filtered and removed in vacuum. The residue was purified by flash chromatography (SiO₂, heptane/EtOAc 1/1) to give the title compound (0.25 g, 31%).

¹H NMR (acetone-d₆): δ 8.47 (1H, s); 8.22 (1H, d); 8.10 (1H, d); 7.83 (1H, t); 6.35 (2H, br s); 4.30 (2H, q); 2.80 (2H, q); 1.35 (3H, t); 1.24 (3H, t).

APCI-MS m/z: 305 [MH⁺].

LC (method A) rt=11.1 min

### b) 5-Amino-3-ethyl-1-(3-nitrophenyl)-1H-pyrazole-4-carboxylic acid

Ethyl 5-amino-3-ethyl-1-(3-nitrophenyl)-1H-pyrazole-4-carboxylate (0.25 g, 0.82 mmol) was dissolved in ethanol (95%, 7 mL) and 3.5 M NaOH (aq) was added (0.82 mL). The solution was refluxed for 18 h. The reaction mixture was cooled to room temperature and carefully acidified (AcOH). Ethyl acetate and water was added, the organic solvent separated and the water was extracted twice with ethyl acetate. The organic solvent was extracted with 1M NaHCO₃ (aq) twice. The basic water phase was acidified with acetic acid and extracted twice with ethyl acetate. The organic solvent was dried over Na₂SO₄, filtered and removed in vacuum, delivering pure title compound (0.15 g, 67%).

¹H NMR (DMSO-d₆): δ 8.38 (1H, t); 8.15 (1H, d); 8.07 (1H, d); 7.77 (1H, t); 2.74 (2H, q); 1.17 (3H, t).

APCI-MS m/z: 277 [MH⁺].

LC (method A) rt=7.1 min

### c) 3-Ethyl-1-(3-nitrophenyl)-7-phenyl-5,6-dihydropyrazolo[3,4-e][1,4]diazepin-4(1H)-one

The pyrazole 5-amino-3-ethyl-1-(3-nitrophenyl)-1H-pyrazole-4-carboxylic acid (0.15 g, 0.54 mmol) was dissolved in dichloromethane (5 mL), and SOCl₂ (1 mL) was added. The mixture was refluxed for 1 h, after which the excess reagent was removed under vacuum. The solid residue was dissolved in THF (5 mL), 2-amino-1-phenylethanone hydrochloride (0.97 g, 5.7 mmol) and triethylamine (2 mL, 14.3 mmol) was added and the mixture stirred at 40° C. for 19 h. The solvent was removed under vacuum, the residue redissolved in ethyl acetate and washed twice with 0.5 M aqueous citric acid. The organic solvent was dried over Na₂SO₄, filtered and removed in vacuum. The residue was purified by preparative HPLC to give the title compound (6 mg, 3%).

¹H NMR (DMSO-d₆): δ 8.66 (1H, t); 8.22 (2H, t); 8.07 (2H, d); 7.97 (1H, t); 7.81 (1H, t); 7.62-7.50 (3H, m); 4.23 (2H, d); 2.92 (2H, q); 1.26 (3H, t).

APCI-MS m/z: 376 [MH⁺].

LC (method A) rt=10.9 min

### EXAMPLE 5

This Example illustrates the preparation of 3-ethyl-1-[4-(methylsulfonyl)phenyl]-7-phenyl-5,6-dihydropyrazolo[3,4-e][1,4]diazepin-4(1H)-one

### a) Ethyl 5-amino-3-ethyl-1-[4-(methylsulfonyl)phenyl]-1H-pyrazole-4-carboxylate

Ethyl 2-cyano-3-ethoxypent-2-enoate (0.51 g, 2.6 mmol), [4-(methylsulfonyl)phenyl]hydrazine hydrochloride (0.68 g, 3.1 mmol) and triethylamine (1.8 ml, 12.9 mmol) were dissolved in methanol (10 mL) and refluxed for 16 h. The solvent was removed under vacuum, after which the residue was redissolved in dichloromethane and washed with water. The organic solvent was dried over Na₂SO₄, filtered and removed in vacuum. The residue was purified by flash chromatography (SiO₂, heptane/EtOAc 1/3) to give the title compound (0.21 g, 24%).

¹H NMR (DMSO-d₆): δ 8.05 (2H, d); 7.84 (2H, d); 4.23 (2H, q); 3.26 (3H, s); 2.72 (2H, q); 1.29 (3H, t); 1.18 (3H, t).

APCI-MS m/z: 338 [MH⁺].

LC (method A) rt=8.8 min

### b) 5-Amino-3-ethyl-1-[4-(methylsulfonyl)phenyl]-1H-pyrazole-4-carboxylic acid

Ethyl 5-amino-3-ethyl-1-[4-(methylsulfonyl)phenyl]-1H-pyrazole-4-carboxylate (0.203 g, 0.6 mmol) was dissolved in ethanol (95%, 5 mL) and 3.5 M NaOH (aq) was added (0.6 mL). The solution was refluxed for 18 h. The reaction mixture was cooled to room temperature and carefully acidified (AcOH). Ethyl acetate and water was added, the organic solvent separated and the water was extracted twice with ethyl acetate. The organic solvent was extracted with 1M NaHCO₃ (aq) twice. The basic water phase was acidified with acetic acid and extracted twice with ethyl acetate. The organic solvent was dried over Na₂SO₄, filtered and removed in vacuum, delivering pure title compound (0.18 g, 97%).

¹H NMR (DMSO-d₆): δ 8.01 (2H, dd); 7.88 (2H, dd); 2.73 (2H, q); 1.88 (3H, s); 1.16 (3H, t).

APCI-MS m/z: 310 [MH⁺].

LC (method A) rt=5.3 min

### c) 3-Ethyl-1-[4-(methylsulfonyl)phenyl]-7-phenyl-5,6-dihydropyrazolo[3,4-e][1,4]diazepin-4(1H)-one

The pyrazole 5-amino-3-ethyl-1-[4-(methylsulfonyl)phenyl]-1H-pyrazole-4-carboxylic acid (0.200 g, 0.65 mmol) was dissolved in dichloromethane (5 mL), and SOCl₂ (1 mL) was added. The mixture was refluxed for 1 h, after which the excess reagent was removed under vacuum. The solid residue was dissolved in THF (5 mL), 2-amino-1-phenylethanone hydrochloride (1.1 g, 6.4 mmol) and triethylamine (0.90 mL, 6.5 mmol) were added and the mixture stirred at 40° C. for 19 h. The solvent was removed under vacuum, the residue redissolved in ethyl acetate and washed twice with 0.5 M aqueous citric acid. The organic solvent was dried over Na₂SO₄, filtered and removed in vacuum. The residue was purified by preparative HPLC to give the title compound (68 mg, 26%).

¹H NMR (acetone-d₆): δ 8.20 (2H, d); 8.17 (2H, d); 8.12-8.09 (2H, m); 7.65-7.50 (3H, m); 7.19 (1H, t); 4.47 (2H, d); 3.19 (3H, s); 3.02 (2H, q); 1.34 (3H, t).

APCI-MS m/z: 410 [MH⁺].

LC (method A) rt=8.2 min

### EXAMPLE 6

This Example illustrates the preparation of 3-Ethyl-1,7-diphenyl-5,6-dihydropyrazolo[3,4-e][1,4]diazepin-4(1H)-one

### a) Ethyl 5-amino-3-ethyl-1-phenyl-1H-pyrazole-4-carboxylate

Ethyl 2-cyano-3-ethoxypent-2-enoate (1.0 g, 5.1 mmol), phenyl hydrazine (583 μl, 5.9 mmol) and triethylamine (3.5 ml, 25.4 mmol) were dissolved in methanol (30 ml) and refluxed for 2 h. The solvent was removed under vacuum, after which the residue was redissolved in dichloromethane and washed with water and a 0.1 M HCl-solution. The organic solvent was dried over MgSO₄, filtered and removed in vacuum to give the crude product (800 mg) which was used in the next step without further purification.

APCI-MS m/z: 260.3 [MH⁺].

### b) 5-Amino-3-ethyl-1-phenyl-1H-pyrazole-4-carboxylic acid

Ethyl 5-amino-3-ethyl-1-phenyl-1H-pyrazole-4-carboxylate (800 mg crude product) was dissolved in ethanol (95%, 30 ml) and 3.5 M NaOH (aq) was added (9 ml). The solution was refluxed for 4 h. Water was added and extracted with ethyl acetate. The basic water phase was neutralized with acetic acid and extracted with ethyl acetate. The organic solvent was dried over MgSO₄, filtered and removed in vacuum, delivering pure title compound (398 mg).

APCI-MS m/z: 274.0 [MH⁺].

### c) 3-Ethyl-1-phenyl-4-[(3H-[1,2,3]triazolo[4,5-b]pyridin-3-yloxy)carbonyl]-1H-pyrazol-5-amine

5-Amino-3-ethyl-1-phenyl-1H-pyrazole-4-carboxylic acid (50 mg, 0.22), HATU (125 mg, 0.33), HOAT (45 mg, 0.33 mmol) and DIEA (113 μl, 0.66 mmol) in dichloromethane (4 ml) were stirred for 1 h. The title compound was obtained by purification on a silica plug (heptane/EtOAc 1:1) (68 mg, 88%).

APCI-MS m/z: 350.3 [MH⁺].

### d) 3-Ethyl-1,7-diphenyl-5,6-dihydropyrazolo[3,4-e][1,4]diazepin-4(1H)-one

3-Ethyl-1-phenyl-4-[(3H-[1,2,3]triazolo[4,5-b]pyridin-3-yloxy)carbonyl]-1H-pyrazol-5-amine (34 mg, 0.10 mmol), 2-amino-1-phenyl-ethanone hydrochloride (27 mg, 0.16 mmol) and Et₃N (52 μl, 0.39 mmol) were stirred in THF (1 ml) at 130° C. for 30 min in microwave. The pure title compound was obtained by preparative HPLC (6 mg, 19%).

¹H NMR (400 MHz, DMSO-d₆) δ 8.03 (2H, d); 7.92 (1H, t); 7.71 (2H, d); 7.62-7.51 (5H, m); 7.41 (1H, t); 4.22 (2H, d); 2.93 (2H, q); 1.27 (3H, t).

APCI-MS m/z: 331.2 [MH⁺].

LC (method A) rt 10.4 min

### EXAMPLE 7

This Example illustrates the preparation of 1-Cyclopentyl-7-(3,4-dimethoxyphenyl)-3-ethyl-5,6-dihydropyrazolo[3,4-e][1,4]diazepin-4(1H)-one.

### a) Ethyl 5-amino-1-cyclopentyl-3-ethyl-1H-pyrazole-4-carboxylate

Ethyl 2-cyano-3-ethoxypent-2-enoate (3.79 g, 0.019 mmol), cyclopentylhydrazine hydrochloride (3.05 g, 0.022 mol) and triethylamine (13.4 ml, 0.096 mol) were dissolved in methanol (100 ml) and refluxed for 5 h. The solvent was removed under vacuum, after which the residue was redissolved in dichloromethane and washed with water. The organic solvent was dried over MgSO₄, filtered and removed in vacuum. The residue was purified by flash chromatography (SiO₂, heptane/EtOAc 8:1) to give the title compound (3.17 g, 66%).

¹H NMR (CDCl₃): δ 5.09 (2H, br s); 4.28 (2H, q); 4.29 (1H, pent); 2.76 (2H, q); 2.15-1.85 (6H, m); 1.73-1.60 (2H, m); 1.35 (3H, t); 1.21 (3H, t).

APCI-MS m/z: 252 [MH⁺].

### b) 5-Amino-1-cyclopentyl-3-ethyl-1H-pyrazole-4-carboxylic acid

Ethyl 1-cyclopentyl-3-ethyl-5-methyl-1H-pyrazole-4-carboxylate (3.17 g, 0.013 mol) was dissolved in ethanol (95%, 28 ml) and 3.5 M NaOH (aq) was added (14 ml). The solution was refluxed for 18 h. Water was added and the mixture was extracted twice with EtOAc. The basic water phase was carefully neutralized with acetic acid and extracted twice with EtOAc. The organic solvent was dried over MgSO₄, filtered and removed in vacuum, delivering pure title compound (2.61 g, 93%).

APCI-MS m/z: 224 [MH⁺].

### c) 1-Cyclopentyl-3-ethyl-4-[(3H-[1,2,3]triazolo[4,5-b]pyridin-3-yloxy)carbonyl]-1H-pyrazol-5-amine

5-Amino-1-cyclopentyl-3-ethyl-1H-pyrazole-4-carboxylic acid (2.61 g, 0.012 mol), HATU (6.80 g, 0.018 mol), HOAT (2.45 g, 0.018) and DIEA (10 ml) in dichloromethane (60 ml) were stirred for 1 h. More dichloromethane was added and the organic phase was washed twice with water. The organic solvent was evaporated and the crude product was purified on a silica plug (heptane/EtOAc 1:1) to give the title compound (2.40 g, 59%).

APCI-MS m/z: 342.1 [MH⁺].

### d) 2-Amino-1-(3,4-dimethoxy-phenyl)-ethanone hydrochloride

2-Bromo-1-(3,4-dimethoxyphenyl)ethanone (518 mg, 2 mmol) was dissolved in acetonitrile (5 ml) together with diformylamide sodium salt (200 mg, 2.1 mmol). The reaction mixture was stirred over night at room temperature. NaBr-salt was removed by filtration and HCl/dioxane (5 ml, 4 M) was added and the mixture was stirred overnight at 50° C. before it was allowed to cool to room temperature. The precipitated product was collected by filtration and washed with dichloromethane three times to give the title compound (291 mg, 63%)

APCI-MS m/z: 196.1 [MH⁺].

### e) 5-Amino-1-cyclopentyl-N-[2-(3,4-dimethoxyphenyl)-2-oxoethyl]-3-ethyl-1H-pyrazole-4-carboxamide

1-Cyclopentyl-3-ethyl-4-[(3H-[1,2,3]triazolo[4,5-b]pyridin-3-yloxy)carbonyl]-1H-pyrazol-5-amine (115 mg, 0.34 mmol), 2-amino-1-(3,4-dimethoxy-phenyl)-ethanone hydrochloride (125 mg, 0.54 mmol) and DIEA (291 μl, 1.7 mmol) in THF (1 ml) were stirred at 70° C. for 4 h. The title compound was obtained by preparative HPLC (66 mg, 48%).

APCI-MS m/z: 401.2 [MH⁺].

### f) 1-Cyclopentyl-7-(3,4-dimethoxyphenyl)-3-ethyl-5,6-dihydropyrazolo[3,4-e][1,4]diazepin-4(1H)-one

5-Amino-1-cyclopentyl-N-[2-(3,4-dimethoxyphenyl)-2-oxoethyl]-3-ethyl-1H-pyrazole-4-carboxamide (66 mg, 0.16 mmol) in xylene was heated to 160° C. and stirred for 1 h. The title compound was obtained by preparative HPLC (38 mg, 60%).

¹H NMR (400 MHz, DMSO-d₆) δ 7.70-7.67 (2H, m); 7.63 (1H, t); 7.13 (1H, d); 5.06 (1H, q); 4.06 (2H, d); 3.87-3.84 (6H, m); 2.81 (2H, q); 2.08-1.93 (4H, m); 1.91-1.81 (2H, m); 1.69-1.61 (2H, m); 1.19 (3H, t).

APCI-MS m/z: 383.3 [MH⁺].

LC (method A) rt 10.2 min

### EXAMPLES 8 to 18

The following compounds were synthesised in an analogous method to Example 7.

| Ex | Compound | ¹H NMR | m/z |
|---|---|---|---|
| 8 | 7-(4-Bromophenyl)-1- | (400 MHz, DMSO-d₆) δ 8.04 (2H, d); | 401.0, 403.0 |
| | cyclopentyl-3-ethyl-5,6- | 7.78 (2H, d); 7.67 (1H, t); 5.08 (1H, q); | |
| | dihydropyrazolo[3,4- | 4.06 (2H, d); 2.82 (2H, q); 2.07-1.83 | |
| | e][1,4]diazepin-4(1H)-one | (6H, m); 1.69-1.62 (2H, m); 1.19 (3H, | |
| | | t). | |
| 9 | 1-Cyclopentyl-3-ethyl-7- | (400 MHz, DMSO-d₆) δ 8.01 (2H, d); | 336.44 |
| | (4-methylphenyl)-5,6- | 7.66 (1H, t); 7.38 (2H, d); 5.08 (1H, q); | |
| | dihydropyrazolo[3,4- | 4.05 (2H, d); 2.81 (2H, q); 2.06-1.83 | |
| | e][1,4]diazepin-4(1H)-one | (6H, m); 1.68-1.62 (2H, m); 1.19 (3H, | |
| | | t). | |
| 10 | 1-Cyclopentyl-3-ethyl-7- | (400 MHz, DMSO-d₆) δ 7.79 (1H, t); | 353.1 |
| | (2-methoxyphenyl)-5,6- | 7.66 (1H, dd); 7.53 (1H, dd); 7.18 (1H, | |
| | dihydropyrazolo[3,4- | d); 7.07 (1H, t); 5.04 (1H, q); 3.94-3.91 | |
| | e][1,4]diazepin-4(1H)-one | (5H, m); 2.82 (2H, q); 2.05-1.79 | |
| | | (6H, m); 1.67-1.58 (2H, m); 1.19 (3H, | |
| | | t). | |
| 11 | Methyl 5-(1-cyclopentyl- | (400 MHz, DMSO-d₆) δ 11.01 (1H, s); | 397.3 |
| | 3-ethyl-4-oxo-1,4,5,6- | 8.44 (1H, d); 8.30 (1H, dd); 7.63 (1H, | |
| | tetrahydropyrazolo[3,4- | t); 7.15 (1H, d); 5.05 (1H, q); 4.04 (2H, | |
| | e][1,4]diazepin-7-yl)-2- | d); 3.93 (3H, s); 2.81 (2H, q); 2.08-1.81 | |
| | hydroxybenzoate | (6H, m); 1.69-1.60 (2H, m); 1.19 | |
| | | (3H, t). | |
| 12 | 7-(4-Chlorophenyl)-1- | (400 MHz, DMSO-d₆) δ 8.12 (2H, d); | 357.2 |
| | cyclopentyl-3-ethyl-5,6- | 7.69-7.62 (3H, m); 5.09 (1H, q); 4.07 | |
| | dihydropyrazolo[3,4- | (2H, d); 2.82 (2H, q); 2.08-1.81 (6H, | |
| | e][1,4]diazepin-4(1H)-one | m); 1.70-1.60 (2H, m); 1.19 (3H, t). | |
| 13 | 7-(3,4-Difluorophenyl)-1- | (400 MHz, DMSO-d₆) δ 8.19 (1H, ddd); | 359.1 |
| | cyclopentyl-3-ethyl-5,6- | 8.00-7.96 (1H, m); 7.69-7.60 (2H, | |
| | dihydropyrazolo[3,4- | m); 5.12 (1H, q); 4.07 (2H, d); 2.82 | |
| | e][1,4]diazepin-4(1H)-one | (2H, q); 2.08-1.80 (6H, m); 1.70-1.60 | |
| | | (2H, m); 1.19 (3H, t). | |
| 14 | 1-Cyclopentyl-7-(2,5- | (400 MHz, DMSO-d₆) δ 7.77 (1H, t); | 383.3 |
| | dimethoxyphenyl)-3-ethyl- | 7.18-7.17 (1H, m); 7.13-7.11 (2H, | |
| | 5,6-dihydropyrazolo[3,4- | m); 5.03 (1H, q); 3.93 (2H, d); 3.86 | |
| | e][1,4]diazepin-4(1H)-one | (3H, s); 3.76 (3H, s); 2.82 (2H, q); | |
| | | 2.07-1.79 (6H, m); 1.68-1.59 (2H, m); | |
| | | 1.19 (3H, t). | |
| 15 | 7-[4-(Benzyloxy)phenyl]- | (400 MHz, DMSO-d₆) δ 8.08 (2H, d); | 429.4 |
| | 1-cyclopentyl-3-ethyl-5,6- | 7.62 (1H, t); 7.48 (2H, d); 7.41 (2H, t); | |
| | dihydropyrazolo[3,4- | 7.38-7.33 (1H, m); 7.19 (2H, d); 5.23 | |
| | e][1,4]diazepin-4(1H)-one | (2H, s); 5.07 (1H, q); 4.04 (2H, d); 2.80 | |
| | | (2H, q); 2.07-1.82 (6H, m); 1.69-1.61 | |
| | | (2H, m); 1.19 (3H, t). | |
| 16 | 1-Cyclopentyl-7-(3,4- | (400 MHz, DMSO-d₆) δ 7.73-7.69 | 395.3 |
| | dihydro-2H-1,5- | (2H, m); 7.59 (1H, t); 7.11 (1H, d); 5.06 | |
| | benzodioxepin-7-yl)-3- | (1H, q); 4.27-4.19 (4H, m); 4.01 (2H, | |
| | ethyl-5,6- | d); 2.80 (2H, q); 2.17 (2H, q); 2.07-1.80 | |
| | dihydropyrazolo[3,4- | (6H, m); 1.69-1.61 (2H, m); 1.18 | |
| | e][1,4]diazepin-4(1H)-one | (3H, t). | |
| 17 | 1-Cyclopentyl-3-ethyl-7- | (400 MHz, DMSO-d₆) δ 8.07 (1H, d); | 313.3 |
| | (2-furyl)-5,6- | 7.68 (1H, t); 7.48 (1H, d); 6.79 (1H, | |
| | dihydropyrazolo[3,4- | dd); 5.01 (1H, q); 4.01 (2H, d); 2.80 | |
| | e][1,4]diazepin-4(1H)-one | (2H, q); 2.05-1.80 (6H, m); 1.68-1.59 | |
| | | (2H, m); 1.18 (3H, t). | |
| 18 | 7-tert-Butyl-1-cyclopentyl- | (400 MHz, DMSO-d₆) δ 7.60 (1H, t); | 303.4 |
| | 3-ethyl-5,6- | 4.90 (1H, q); 3.61 (2H, d); 2.76 (2H, q); | |
| | dihydropyrazolo[3,4- | 2.02-1.77 (6H, m); 1.66-1.57 (2H, | |
| | e][1,4]diazepin-4(1H)-one | m); 1.21 (9H, s); 1.16 (3H, t). | |

### EXAMPLE 19

This Example illustrates the preparation of 1-Cyclopentyl-3-ethyl-7-(3-methoxyphenyl)-5,6-dihydropyrazolo[3,4-e][1,4]diazepin-4(1H)-one

1-Cyclopentyl-3-ethyl-4-[(3H-[1,2,3]thiazolo[4,5-b]pyridin-3-yloxy)carbonyl]-1H-pyrazol-5-amine (124 mg, 0.36 mmol), 2-amino-1-(3-methoxy-phenyl)-ethanone hydrochloride (117 mg, 0.58 mmol) and Et₃N (251 μl, 1.8 mmol) in THF (1 ml) were stirred at 120° C. for 20 min in microwave. The title compound was obtained by preparative HPLC (40 mg, 32%).

¹H NMR (400 MHz, DMSO-d₆) δ 7.70-7.66 (2H, m); 7.63-7.61 (1H, m); 7.49 (1H, t); 7.19 (1H, dd); 5.08 (1H, q); 4.06 (2H, d); 3.85 (3H, s); 2.82 (2H, q); 2.08-1.92 (4H, m); 1.90-1.81 (2H, m); 1.69-1.61 (2H, m); 1.19 (3H, t).

91-1.81 (2H, m); 1.69-1.61 (2H, m); 1.19 (3H, t).

APCI-MS m/z: 353.1 [MH⁺].

LC (method A) rt 11.4 min

### EXAMPLES 20 to 26

The following compounds were synthesised in an analogous method to Example 19.

| Ex | Compound | ¹H NMR | m/z |
|---|---|---|---|
| 20 | Methyl 3-(1-cyclopentyl- | (400 MHz, DMSO-d₆) δ 7.52 (1H, t); | 333.1 |
| | 3-ethyl-4-oxo-1,4,5,6- | 4.82 (1H, q); 3.59 (3H, s); 3.55 (2H, | |
| | tetrahydropyrazolo[3,4- | d); 2.92 (2H, t); 2.76 (2H, q); 2.67 | |
| | e][1,4]diazepin-7- | (2H, t); 1.98-1.77 (6H, m); 1.66-1.54 | |
| | yl)propanoate | (2H, m); 1.15 (3H, t). | |
| 21 | 1-Cyclopentyl-3-ethyl-7- | (300 MHz, DMSO-d₆) δ 8.18 (2H, | 341.1 |
| | (4-fluorophenyl)-5,6- | ddd); 7.66 (1H, t); 7.45-7.38 (2H, m); | |
| | dihydropyrazolo[3,4- | 5.08 (1H, q); 4.07 (2H, d); 2.82 (2H, | |
| | e][1,4]diazepin-4(1H)-one | q); 2.09-1.59 (8H, m); 1.19 (3H, t). | |
| 22 | 1-Cyclopentyl-3-ethyl-7- | (400 MHz, DMSO-d₆) δ 8.23 (2H, d); | 407.1 |
| | [4-(trifluoromethoxy)phenyl]- | 7.68 (1H, t); 7.56 (2H, d); 5.09 (1H, | |
| | 5,6-dihydropyrazolo[3,4- | q); 4.09 (2H, d); 2.82 (2H, q); 2.09-1.79 | |
| | e][1,4]diazepin-4(1H)-one | (6H, m); 1.70-1.60 (2H, m); | |
| | | 1.19 (3H, t). | |
| 23 | 4-(1-Cyclopentyl-3-ethyl- | (400 MHz, DMSO-d₆) δ 8.26 (2H, d); | 348.1 |
| | 4-oxo-1,4,5,6- | 8.05 (2H, d); 7.70 (1H, t); 5.11 (1H, | |
| | tetrahydropyrazolo[3,4- | q); 4.10 (2H, d); 2.83 (2H, q); 2.09-1.80 | |
| | e][1,4]diazepin-7- | (6H, m); 1.71-1.61 (2H, m); | |
| | yl)benzonitrile | 1.20 (3H, t). | |
| 24 | 1-Cyclopentyl-3-ethyl-7- | (400 MHz, DMSO-d₆) δ 8.32 (2H, d); | 401.1 |
| | [4-(methylsulfonyl)phenyl]- | 8.10 (2H, d); 7.72 (1H, t); 5.11 (1H, | |
| | 5,6-dihydropyrazolo[3,4- | q); 4.12 (2H, d); *(3H in solvent | |
| | e][1,4]diazepin-4(1H)-one | peak); 2.83 (2H, q); 2.09-1.82 (6H, | |
| | | m); 1.70-1.61 (2H, m); 1.20 (3H, t). | |
| 25 | 1-Cyclopentyl-3-ethyl-7- | (300 MHz, DMSO-d₆) δ 8.21 (1H, d); | 337.1 |
| | methyl-6-phenyl-5,6- | 7.25-7.10 (5H, m); 5.09 (1H, t); 4.86 | |
| | dihydropyrazolo[3,4- | (1H, q); 3.29 (3H, s); 2.69-2.57 (2H, | |
| | e][1,4]diazepin-4(1H)-one | m); 2.00-1.35 (8H, m); 0.97 (3H, t). | |

### EXAMPLE 26

This Example illustrates the preparation of 1-Cyclopentyl-3-ethyl-5-methyl-7-phenyl-5,6-dihydropyrazolo[3,4-e][1,4]diazepin-4(1H)-one

1-Cyclopentyl-3-ethyl-7-phenyl-5,6-dihydropyrazolo[3,4-e][1,4]diaz epin-4(1H)-one (10 mg, 0.031 mmol), methyl iodide (24 mg, 0.074 mmol) and NaH (50% in oil, 3.6 mg, 0.074) in THF (0.5 ml) were stirred at room temperature for 3 h. The title compound was obtained by preparative HPLC (12 mg, 48%).

¹H NMR (400 MHz cd3od) δ 8.22 (2H, dd); 7.63-7.55 (3H, m); 5.17 (1H, q); 4.37 (2H, s); 3.17 (3H, s); 2.93 (2H, q); 2.16-1.90 (6H, m); 1.78-1.68 (2H, m); 1.28 (3H, t).

APCI-MS m/z: 337.3 [MH⁺].

LC (method A) rt 12.3 min

### EXAMPLE 27

This Example illustrates the preparation of 5-(1-Cyclopentyl-3-ethyl-4-oxo-1,4,5,6-tetrahydropyrazolo[3,4-e][1,4]diazepin-7-yl)-2-hydroxybenzoic acid

Methyl 5-(1-cyclopentyl-3-ethyl-4-oxo-1,4,5,6-tetrahydropyrazolo[3,4-e][1,4]diazepin-7-yl)-2-hydroxybenzoate (7 mg, 0.018 mmol) and lithium hydroxide (2 mg, 0.087 mmol) in THF/water 4:1 (2.5 ml) were stirred at 60° C. over night. The title compound was obtained by preparative HPLC (1.7 mg, 25%).

¹H NMR (400 MHz, DMSO-d₆) δ 8.40 (1H, d); 8.04 (1H, dd); 7.60 (1H, t); 6.72 (1H, d); 5.05 (1H, q); 4.00 (2H, d); 2.79 (2H, q); 2.06-1.82 (6H, m); 1.68-1.61 (2H, m); 1.18 (3H, t).

APCI-MS m/z: 383.0 [MH⁺].

LC (method A) rt 9.8 min

### EXAMPLE 28

This Example illustrates the preparation of 3-(1-Cyclopentyl-3-ethyl-4-oxo-1,4,5,6-tetrahydropyrazolo[3,4-e][1,4]diazepin-7-yl)propanoic acid

Methyl 3-(1-cyclopentyl-3-ethyl-4-oxo-1,4,5,6-tetrahydropyrazolo[3,4-e][1,4]diazepin-7-yl)propanoate (62 mg, 0.19 mmol) and lithium hydroxide (4.5 mg, 0.19 mmol) in methanol/water 9:1 (3.3 ml) were stirred at 60° C. for 2 h. Water was added and the mixture was extracted with EtOAc. The water phase was neutralized with HCl and extracted with EtOAc. The organic solvent was dried over MgSO₄, filtered and removed in vacuum, delivering pure title compound (36 mg, 60%).

APCI-MS m/z: 319.1 [MH⁺].

LC (method A) rt 6.0 min

### EXAMPLE 29

This Example illustrates the preparation of 3-(1-Cyclopentyl-3-ethyl-4-oxo-1,4,5,6-tetrahydropyrazolo[3,4-e][1,4]diazepin-7-yl)-N-[2-(dimethylamino)ethyl]propanamide

3-(1-Cyclopentyl-3-ethyl-4-oxo-1,4,5,6-tetrahydropyrazolo[3,4-e][1,4]diazepin-7-yl)propanoic acid (30 mg, 0.094 mmol), N,N-dimethylethylenediamine (16 μl, 0.141 mmol), HATU (53 mg, 0.14 mmol), HOAT (19 mg, 0.14 mmol) and DIEA (48 μl, 0.28 mmol) in dichloromethane were stirred for 1 h. The title compound was obtained by preparative HPLC (10 mg, 27%).

¹H NMR (400 MHz, DMSO-d₆) δ 7.35 (1H, t); 6.81 (1H, s); 4.49 (1H, q); 3.43 (1H, dd); 3.20 (2H, t); 3.08 (1H, dd); 2.73-2.58 (2H, m); 2.45-2.19 (5H, m); 2.18-2.06 (7H, m); 1.97-1.74 (6H, m); 1.63-1.51 (2H, m); 1.08 (3H, t).

APCI-MS m/z: 389.2 [MH⁺].

LC (method A) rt 4.6 min

### EXAMPLE 30

This Example illustrates the preparation of 1-Cyclopentyl-3-ethyl-7-phenyl-5,6,7,8-tetrahydropyrazolo[3,4-e][1,4]diazepin-4(1H)-one

1-Cyclopentyl-3-ethyl-7-phenyl-5,6-dihydropyrazolo[3,4-e][1,4]diazepin-4(1H)-one (62 mg, 0.19 mmol) and NaCNBH₃ (sodium cyanoborohydride; 24 mg, 0.38 mmol) were stirred in a mixture of methanol (2 ml) and acetic acid (2 drops) over night at r t. The title compound was obtained by preparative HPLC (44 mg, 71%).

¹H NMR (400 MHz, DMSO-d₆) δ 7.37-7.32 (2H, m); 7.28-7.24 (3H, m); 6.98-6.88 (1H, m); 4.68 (1H, d); 4.51 (1H, t); 3.53-3.45 (1H, m); *(1H in solvent peak); 2.75-2.58 (2H, m); 1.99-1.73 (6H, m); 1.61-1.51 (2H, m); 1.07 (3H, t).

APCI-MS m/z: 325.3 [MH⁺].

LC (method A) rt 7.6 min

### EXAMPLE 31

This Example illustrates the preparation of 3-(Methylthio)-1,7-diphenyl-5,6-dihydropyrazolo[3,4-e][1,4]diazepin-4(1H)-one.

### a) Ethyl 5-amino-3-(methylthio)-1-phenyl-1H-pyrazole-4-carboxylate

2-Cyano-3,3-bis-methylsulfanyl-acrylic acid ethyl ester (1 g, 4.6 mmol), phenyl hydrazine (0.5 g, 4.6 mmol) were refluxed in isopropanol for 4 h. The isopropanol was evaporated and the crude product was directly used in the next step without further purification.

APCI-MS m/z: 278.0 [MH⁺].

### b) 5-Amino-3-(methylthio)-1-phenyl-1H-pyrazole-4-carboxylic acid

Ethyl 5-amino-3-(methylthio)-1-phenyl-1H-pyrazole-4-carboxylate (700 mg, 2.5 mmol) and lithium hydroxide (483 mg, 20.2 mmol) were stirred at reflux in methanol/water 10:1 (35 ml) over night. The methanol was evaporated, water was added and the mixture was acidified with acetic acid. The mixture was extracted twice with dichloromethane. The organic solvent was dried over MgSO₄, filtered and removed in vacuum, delivering pure title compound (380 mg, 60%).

APCI-MS m/z: 250.0 [MH⁺].

### c) 3-(Methylthio)-1-phenyl-4-[(3H-[1,2,3]triazolo[4,5-b]pyridin-3-yloxy)carbonyl]-1H-pyrazol-5-amine

5-Amino-3-(methylthio)-1-phenyl-1H-pyrazole-4-carboxylic acid (151 mg, 0.61 mmol), HATU (346 mg, 0.91 mmol), HOAT (124 mg, 0.91 mmol) and DIEA (314 μl, 1.8 mmol) in dichloromethane (3 ml) were stirred for 1 h. More dichloromethane was added and the organic phase was washed with water, dried and removed in vacuum delivering the title compound (217 mg, 96%).

APCI-MS m/z: 368.0 [MH⁺].

### d) 5-Amino-3-(methylthio)-N-(2-oxo-2-phenylethyl)-1-phenyl-1H-pyrazole-4-carboxamide

3-(ethylthio)-1-phenyl-4-[(3H-[1,2,3]triazolo[4,5-b]pyridin-3-yloxy)carbonyl]-1H-pyrazol-5-amine (94 mg, 0.26 mmol), 2-amino-1-phenyl-ethanone hydrochloride (70 mg, 0.41 mmol), and Et₃N (178 μl, 1.3 mmol) in THF (1 ml) were stirred at 120° C. for 40 min in microwave. The title compound was obtained by preparative HPLC (31 mg, 33%).

APCI-MS m/z: 367.0 [MH⁺].

### e) 3-(Methylthio)-1,7-diphenyl-5,6-dihydropyrazolo[3,4-e][1,4]diazepin-4(1H)-one

5-Amino-3-(methylthio)-N-(2-oxo-2-phenylethyl)-1-phenyl-1H-pyrazole-4-carboxamide (31 mg, 0.08 mmol) was stirred in xylene (1 ml) at 160° C. for 2 h. The title compound was obtained by preparative HPLC (5 mg, 18%).

¹H NMR (400 MHz, CD₃OD) δ 8.08 (2H, m); 7.74 (2H, dd); 7.60-7.50 (5H, m); 7.42 (1H, t); 4.33 (2H, s); 2.61 (3H, s).

APCI-MS m/z: 349.3 [MH⁺].

LC (method A) rt 11.0 min

### EXAMPLE 32

This Example illustrates the preparation of 1-(1,1-Dioxidotetrahydrothien-3-yl)-3-ethyl-7-phenyl-5,6-dihydropyrazolo[3,4-e][1,4]diazepin-4(1H)-one

### a) Ethyl 5-amino-1-(1,1-dioxidotetrahydrothiophen-3-yl)-3-ethyl-1H-pyrazole-4-carboxylate

Ethyl 2-cyano-3-ethoxypent-2-enoate (638 mg, 3.2 mmol), (1,1-Dioxo-tetrahydrothiophen-3-yl)-hydrazine hydrochloride (700 mg, 3.8 mmol) and triethylamine (2.3 ml, 16.2 mmol) were dissolved in methanol (10 ml) and refluxed for 16 h. The solvent was removed under vacuum, after which the residue was redissolved in dichloromethane and washed with water. The organic solvent was dried over MgSO₄, filtered and the solvent removed in vacuum to give the crude product which was used in the next step without further purification (833 mg, 66%).

APCI-MS m/z: 302.1 [MH⁺].

### b) 5-Amino-1-(1,1-dioxidotetrahydrothiophen-3-yl)-3-ethyl-1H-pyrazole-4-carboxylic acid

Ethyl 5-amino-1-(1,1-dioxidotetrahydrothien-3-yl)-3-ethyl-1H-pyrazole-4-carboxylate (773 mg, 2.6 mmol) and lithium hydroxide (490 mg, 20.5 mmol) were stirred at reflux in methanol/water 10:1 (35 ml) over night. Water was added and the mixture was extracted with EtOAc. The aqueous phase was neutralized with 0.5 M citric acid and extracted with EtOAc. The organic solvent was dried over MgSO₄, filtered and removed in vacuum, delivering the title compound with 70% purity (323 mg, 46%).

APCI-MS m/z: 274.0 [MH⁺].

### c) 1-(1,1-Dioxidotetrahydrothiophen-3-yl)-3-ethyl-4-[(3H-[1,2,3]triazolo[4,5-b]pyridin-3-yloxy)carbonyl]-1H-pyrazol-5-amine

5-Amino-1-(1,1-dioxidotetrahydrothien-3-yl)-3-ethyl-1H-pyrazole-4-carboxylic acid (60 mg, 0.22), HATU (125 mg, 0.33), HOAT (45 mg, 0.33 mmol) and DIEA (113 μl, 0.66 mmol) in dichloromethane (4 ml) were stirred for 1 h. Additional dichloromethane was added and the organic phase was washed with water, dried and removed in vacuum delivering the crude product which was used in the next step without further purification (53 mg).

APCI-MS m/z: 392.0 [MH⁺].

### d) 1-(1,1-Dioxidotetrahydrothiophen-3-yl)-3-ethyl-7-phenyl-5,6-dihydropyrazolo[3,4-e][1,4]diazepin-4(1H)-one

1-(1,1-Dioxidotetrahydrothien-3-yl)-3-ethyl-4-[(3H-[1,2,3]triazolo[4,5-b]pyridin-3-yloxy)carbonyl]-1H-pyrazol-5-amine (53 mg, 0.14 mmol), 2-amino-1-phenyl-ethanone hydrochloride (37 mg, 0.22 mmol) and Et₃N (94 μl, 0.68 mmol) were stirred in THF (1 ml) at 120° C. for 20 min in microwave. The pure title compound was obtained by preparative HPLC (6 mg, 12%).

¹H NMR (400 MHz, DMSO-d₆) δ 8.17 (2H, m); 7.78 (1H, t); 7.65-7.55 (3H, m); 5.64 (1H, q); 4.12 (2H, d); 3.71 (1H, dd); 3.53-3.41 (2H, m); *(2H in solvent peak); 2.85 (2H, q); 2.61-2.55 (1H, m); 1.22 (3H, t).

APCI-MS m/z: 373.1 [MH⁺].

LC (method A) rt 7.1 min

### EXAMPLE 33

This Example illustrates the preparation of 3-Ethyl-7-phenyl-1-tetrahydro-2H-thiopyran-4-yl-5,6-dihydropyrazolo[3,4-e][1,4]diazepin-4(1H)-one

### a) (Tetrahydro-thiopyran-4-yl)-hydrazine trifluoroacetate

Tetrahydro-thiopyran-4-one (1.3 g, 0.011 mmol) and tert-butyl carbazate (1.5 g, 0.011 mmol) in methanol were stirred at r t for 1.5 h. The solvent was removed in vacuum. The remaining solid was dissolved in acetic acid to which NaCNBH₃ was added (0.7 g, 0.011). The mixture was stirred for 1 h, neutralized with 1M NaOH and extracted with dichloromethane. The organic phase was washed with a saturated solution of NaHCO₃, dried over MgSO₄ and evaporated. To the solid crude dichloromethane and TFA were added 1:1 (20 ml) and the mixture was stirred. The title compound was obtained after 2 h when the solvent was finally removed in vacuum (2.3 g, 85%).

APCI-MS m/z: 133.1 [MH⁺].

### b) Ethyl 5-amino-3-ethyl-1-tetrahydro-2H-thiopyran-4-yl-1H-pyrazole-4-carboxylate

Ethyl 2-cyano-3-ethoxypent-2-enoate (1.6 g, 8.0 mmol), tetrahydro-thiopyran-4-yl)-hydrazine trifluoroacetate (2.3 g, 9.3 mmol) and Et₃N (5.6 ml, 40 mmol) were refluxed in methanol (50 ml) for 1 h. The solvent was evaporated. dichloromethane was added and the mixture was washed twice with water. The title compound was obtained after removal of the organic solvent in vacuum (2.5 g, 94%).

APCI-MS m/z: 284.1 [MH⁺].

### c) 5-Amino-3-ethyl-1-tetrahydro-2H-thiopyran-4-yl-1H-pyrazole-4-carboxylic acid

Ethyl 5-amino-3-ethyl-1-tetrahydro-2H-thiopyran-4-yl-1H-pyrazole-4-carboxylate (1.2 g, 4.23 mmol) and lithium hydroxide (0.5 g, 21.1 mmol) in methanol/water 8:1 (28 ml) were refluxed over night. Water was added and the mixture vas extracted with EtOAc. The aqueous phase was neutralized with HCl and extracted with EtOAc. The organic phase was dried over MgSO₄ and evaporated to give the title compound with 75% purity (680 mg, 63%).

APCI-MS m/z: 256.1 [MH⁺].

### d) 3-Ethyl-1-tetrahydro-2H-thiopyran-4-yl-4-[(3H-[1,2,3]triazolo[4,5-b]pyridin-3-yloxy)carbonyl]-1H-pyrazol-5-amine

5-Amino-3-ethyl-1-tetrahydro-2H-thiopyran-4-yl-1H-pyrazole-4-carboxylic acid (0.68 g, 2.7 mmol), HATU (1.52 g, 4.0 mmol), HOAT (0.54 g, 4.0 mmol) and DIEA (1.4 ml, 8.0 mmol) in dichloromethane were stirred for 2 h. The mixture was partioned between dichloromethane and water and the organic phase was washed with a saturated solution of NaHCO₃ The crude product was purified on a silica plug (heptane/EtOAc 1:1) to give the title compound (0.87 g, 88%).

APCI-MS m/z: 374.3 [MH⁺].

### e) 3-Ethyl-7-phenyl-1-tetrahydro-2H-thiopyran-4-yl-5,6-dihydropyrazolo[3,4-e][1,4]diazepin-4(M1)-one

3-Ethyl-1-tetrahydro-2H-thiopyran-4-yl-4-[(3H-[1,2,3]triazolo[4,5-b]pyridin-3-yloxy)carbonyl]-1H-pyrazol-5-amine (200 mg, 0.54 mmol), 2-amino-1-phenyl-ethanone hydrochloride (148 mg, 0.86 mmol) and Et₃N (374 μl, 2.68 mmol) were stirred in THF (2 ml) at 120° C. for 25 min in microwave. The pure title compound was obtained by preparative HPLC (77 mg, 40%).

¹H NMR (400 MHz, DMSO-d₆) δ 8.12 (2H, m); 7.71 (1H, t); 7.63-7.55 (3H, m); 4.60 (1H, q); 4.08 (2H, d); 2.94-2.79 (4H, m); 2.77-2.69 (2H, m); 2.19-2.13 (4H, m); 1.20 (3H, t).

APCI-MS m/z: 355.1 [MH⁺].

LC (method A) rt 9.9 min

### EXAMPLE 34 and 35

This Example illustrates the preparation 3-Ethyl-1-(1-oxidotetrahydro-2H-thiopyran-4-yl)-7-phenyl-5,6-dihydropyrazolo[3,4-e][1,4]diazepin-4(1H)-one and 1-(1,1-Dioxidotetrahydro-2H-thiopyran-4-yl)-3-ethyl-7-phenyl-5,6-dihydropyrazolo[3,4-e][1,4]diazepin-4(1H)-one.

3-Ethyl-7-phenyl-1-tetrahydro-2H-thiopyran-4-yl-5,6-dihydropyrazolo[3,4-e][1,4]diazepin-4(1H)-one (20 mg, 0.056 mmol) was dissolved in THF (2 ml) and cooled on an ice bath. MCPBA (70%, 22 mg, 0.089 mmol) dissolved in THF was added drop wise. The mixture was stirred for 3 h. The sulfoxide and the sulfone were formed in a 3:7 ratio. The title compounds were obtained by preparative HPLC (6 mg, 29% and 9 mg, 42

Data for 3-Ethyl-1-(1-oxidotetrahydro-2H-thiopyran-4-yl)-7-phenyl-5,6-dihydropyrazolo[3,4-e][1,4]diazepin-4(1H)-one:

¹H NMR (400 MHz, DMSO-d₆) δ 8.15 (2H, m); 7.74 (1H, t); 7.64-7.55 (3H, m); 4.93 (1H, q); 4.09 (2H, d); *(2H in solvent peak); 2.97 (2H, t); 2.82 (2H, q); 2.34-2.10 (4H, m); 1.20 (3H, t).

APCI-MS m/z: 371.1 [MH⁺].

LC (method A) rt 5.0 min

Data for 1-(1,1-Dioxidotetrahydro-2H-thiopyran-4-yl)-3-ethyl-7-phenyl-5,6-dihydropyrazolo[3,4-e][1,4]diazepin-4(1H)-one:

¹H NMR (400 MHz, DMSO-d₆) δ 8.17 (2H, m); 7.74 (1H, t); 7.65-7.54 (3H, m); 5.03 (1H, t); 4.10 (2H, d); 3.52 (2H, t); 3.21 (2H, d); 2.84 (2H, q); 2.61-2.53 (2H, m); 2.22 (2H, d); 1.21 (3H, t).

APCI-MS m/z: 387.1 [MH⁺].

LC (method A) rt 6.6 min

### EXAMPLE 36

This Example illustrates the preparation of 1-Cyclopentyl-3-methyl-7-phenyl-5,6-dihydropyrazolo[3,4-e][1,4]diazepin-4(1H)-one

### a) Ethyl (2E)-2-cyano-3-ethoxybut-2-enoate

Ethyl cyanoacetate (15 ml, 0.14 mol) and triethyl orthoacetate (25 ml, 0.14 mol) in acetic anhydride (100 ml) were heated at 90° C. over night. The solvent was removed in vacuum and the remaining liquid was put in the freezer. The formed precipitate was filtered off to give the pure title compound (7.26 g, 28%).

APCI-MS m/z: 184.1 [MH⁺].

### b) Ethyl 5-amino-1-cyclopentyl-3-methyl-1H-pyrazole-4-carboxylate

Ethyl (2E)-2-cyano-3-ethoxybut-2-enoate (2.24 g, 0.012 mol), cyclopentylhydrazine hydrochloride (1.94 g, 0.014 mol) and Et₃N (8.2 ml, 0.061 mol) in methanol (100 ml) were heated to reflux for 2 h. The methanol was removed in vacuum and dichloromethane was added. The mixture was washed twice with water and once with a 0.1% HCl-solution. The organic solvent was dried over MgSO₄, filtered and evaporated. The residue was purified on silica (heptane/EtOAc) to give the title compound (2.73 g, 94%).

APCI-MS m/z: 238.1 [MH⁺].

### c) 5-Amino-1-cyclopentyl-3-methyl-1H-pyrazole-4-carboxylic acid

Ethyl 5-amino-1-cyclopentyl-3-methyl-1H-pyrazole-4-carboxylate (1.5 g, 6.3 mmol) was refluxed over night in a mixture of a 3.5 M solution of NaOH (6 ml) and ethanol (12 ml). Water was added and the mixture was extracted with EtOAc. The aqueous phase was neutralized with HCl and extracted with EtOAc. The organic phase was dried over MgSO₄, filtered and evaporated to give the title compound with 20% impurity (0.9 g, 70%).

APCI-MS m/z: 210.1 [MH⁺].

### d) 1-Cyclopentyl-3-methyl-4-[(3H-[1,2,3]triazolo[4,5-b]pyridin-3-yloxy)carbonyl]-1H-pyrazol-5-amine

5-Amino-1-cyclopentyl-3-methyl-1H-pyrazole-4-carboxylic acid (0.9 g, 4.4 mmol), HATU (2.5 g, 6.7 mmol), HOAT (0.9 g, 6.7 mmol) and DIEA (2.3 ml, 13.3 mmol) in dichloromethane (40 ml) were stirred for 1 h. The title compound was obtained by purification on a silica plug (heptane/EtOAc 3:2) (1.2 g, 86%).

APCI-MS m/z: 328.1 [MH⁺].

### e) 1-Cyclopentyl-3-methyl-7-phenyl-5,6-dihydropyrazolo[3,4-e][1,4]diazepin-4(1H)-one

1-Cyclopentyl-3-methyl-4-[(3H-[1,2,3]triazolo[4,5-b]pyridin-3-yloxy)carbonyl]-1H-pyrazol-5-amine (207 mg, 0.63 mmol)), 2-amino-1-phenyl-ethanone hydrochloride (174 mg, 1.01 mmol) and Et₃N (440 μl, 3.16 mmol) were stirred in THF (2 ml) at 120° C. for 30 min in microwave. The pure title compound was obtained by preparative HPLC (100 mg, 51%).

¹H NMR (300 MHz, DMSO-d₆) δ 8.13-8.09 (2H, m); 7.70 (1H, t); 7.64-7.53 (3H, m); 5.10 (1H, q); 4.09 (2H, d); 2.38 (3H, s); 2.09-1.59 (8H, m).

APCI-MS m/z: 309.1 [MH⁺].

LC (method A) rt 9.7 min

### EXAMPLE 37

### Human Phosphodiesterase B2 Alpha Screen Assay

The assay uses recombinant Human Phosphodiesterase B2 (PDE4B2) produced in house (PrAZL0133), stored at −20° C. The substrate uses cAMP, part of the Alpha Screen cAMP kit (Perkin Elmer, Cat #6760625M), stored at 4° C. The Alpha Screen kit also includes biotinylated cAMP, acceptor and donor beads.

The assay additions were as follows: Test compounds and controls were added to white 384-well flat-bottom plates (Greiner, Cat #781075), 0.2 μl in 100% DMSO, followed by 10 μl PDE4B2 in reaction buffer. The reaction buffer constitution was: 50 mM Tris (pH 7.5), 8.3 mM MgCl2, 1.7 mM EGTA and 0.01% (w/v) Brij®35. The enzyme and the compounds were incubated at room temperature for 15 minutes. Then 10 μl cAMP in reaction buffer was added. The assay was stopped after 60 minutes incubation at room temperature by adding 10 μl acceptor beads in detection buffer with 40 mM EDTA. The detection buffer constitution was: 5 mM Tris (pH 7.5), 0.1% (w/v) BSA and 0.1% (v/v) Tween 20. This addition followed by an addition of 10 μl donor beads in detection buffer, with biotinylated cAMP. The plates were then incubated, dark at room temperature, for 5 hours followed by measurement on a Fusion™-α analyser. IC₅₀ values (presented in Table 1) were determined using Xlfit curve fitting using model 205.

TABLE 1

| Example | PDE4B2 IC50 (nM) |
|---|---|
| 1 | 0.33 |
| 2 | 0.98 |
| 3 | 6.1 |
| 4 | 4.8 |
| 5 | 43 |
| 6 | 11.0 |
| 7 | 1.47 |
| 8 | 0.84 |
| 9 | 0.61 |
| 10 | 0.91 |
| 11 | 0.59 |
| 12 | 0.57 |
| 13 | 0.73 |
| 14 | 0.50 |
| 15 | 0.90 |
| 16 | 0.43 |
| 17 | 2.16 |
| 18 | 0.53 |
| 19 | 0.55 |
| 20 | 28.7 |
| 21 | 1.05 |
| 22 | 4.16 |
| 23 | 1.21 |
| 24 | 3.92 |
| 25 | 385.0 |
| 26 | 103.0 |
| 27 | 0.52 |
| 28 | 65.3 |
| 29 | 4480.0 |
| 30 | 158.0 |
| 31 | 426.0 |
| 32 | 5.8 |
| 33 | 3.9 |
| 34 | 99.1 |
| 35 | 40.3 |
| 36 | 9.49 |

1. A compound of formula (I): 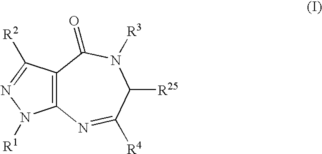
wherein:
R¹ is a 5- or 6-membered, saturated or unsaturated, carbocyclic or heterocyclic ring; when
R¹ is aryl or heteroaryl then said aryl or heteroaryl is optionally substituted as recited below; when R¹ is a non-aromatic carbocycle or non-aromatic heterocyclyl then said ring is optionally substituted by C₁₋₆ alkyl;
R² is C₁₋₄ alkyl, C₁₋₄ alkoxy or C₁₋₄ alkylthio;
R³ is hydrogen or C₁₋₄ alkyl;
R⁴ is C₁₋₆ alkyl (optionally substituted by CO₂R²⁶ or C(O)NR²⁷R²⁸), optionally substituted aryl or optionally substituted heteroaryl;
R²⁵ is hydrogen or optionally substituted phenyl;
the foregoing aryl, heteroaryl and phenyl rings are, independently, optionally substituted by: halogen, cyano, nitro, hydroxy, S(O)_{q}R⁵, OC(O)NR⁶R⁷, NR⁸R⁹, NR¹⁰C(O)R¹¹, NR¹²C(O)NR¹³R¹⁴, S(O)₂NR¹⁵R¹⁶, NR¹⁷S(O)₂R¹⁸, C(O)NR¹⁹R²⁰, C(O)R²¹, CO₂R²², NR²³CO₂R²⁴, C₁₋₆ alkyl, C₁₋₆ haloalkyl, C₁₋₆ alkoxy(C₁₋₆)alkyl, C₁₋₆ alkoxy, C₁₋₆ haloalkoxy, C₁₋₆ alkoxy(C₁₋₆)alkoxy, C₁₋₆ alkylthio, O(CH₂)ₙO, phenyl(C₁₋₆)alkyl, phenoxy(C₁₋₆)alkyl or phenyl(C₁₋₆)alkoxy; the phenyl rings of the last three substituents being optionally substituted by halogen, C₁₋₄ alkyl, C₁₋₄ alkoxy, CF₃ or OCF₃;
n is 1, 2 or 3;
q is 0, 1 or 2;
R⁶ and R⁷, R¹³ and R¹⁴, R¹⁵ and R¹⁶, or R¹⁹ and R²⁰ can join to form, with the nitrogen to which they are bonded, a 5- or 6-membered ring;
R⁵, R⁶, R⁷, R⁸, R⁹, R¹⁰, R¹¹, R¹², R¹³, R¹⁴, R¹⁵, R¹⁶, R¹⁷, R¹⁸, R¹⁹, R²⁰, R²¹, R²², R²³, R²⁴ and R²⁶ are, independently, C₁₋₆ alkyl (optionally substituted by halogen, hydroxy or C₁₋₆ alkoxy) or phenyl (itself optionally substituted by halogen, C₁₋₄ alkyl, C₁₋₄ alkoxy, CF₃ or OCF₃);
R⁶R⁷, R⁸, R⁹, R¹⁰, R¹¹, R¹², R¹³, R¹⁴, R¹⁵, R¹⁶, R¹⁷, R¹⁹, R²⁰, R²¹, R²², R²³ and R²⁶ can also be hydrogen;
R²⁷ is hydrogen or C₁₋₆ alkyl;
R²⁸ is hydrogen or C₁₋₆ alkyl (optionally substituted by NH₂, NH(C₁₋₆ alkyl) or N(C₁₋₆ alkyl)₂);
or a N-oxide thereof; or a pharmaceutically acceptable salt thereof.

2. A compound of formula (I) as claimed in claim 1 wherein R² is C₁₋₄ alkyl.

3. A compound of formula (I) as claimed in claim 1 wherein R³ is hydrogen.

4. A compound of formula (I) as claimed in claim 1 wherein R²⁵ is hydrogen.

5. A compound of formula (I) as claimed in claim 1 wherein R¹ is cyclopentyl, or phenyl (optionally substituted as described in claim 1).

6. A compound of formula (I) as claimed in claim 1 wherein R⁴ is phenyl optionally substituted by: halogen, cyano, nitro, S(O)_{q}R⁵, NHC(O)R¹¹, S(O)₂NR¹⁵R¹⁶, CO₂R²², C₁₋₆ alkyl, C₁₋₆ haloalkyl, C₁₋₆ alkoxy or C₁₋₆ haloalkoxy; wherein q is 0, 1 or 2; R⁵ is C₁₋₆ alkyl; R¹¹, R¹⁵, R¹⁶ and R²² are, independently, hydrogen or C₁₋₆ alkyl.

7. A compound of formula (I) which is:
1-Cyclopentyl-3-ethyl-7-phenyl-5,6-dihydropyrazolo[3,4-e][1,4]diazepin-4(1H)-one;
1-Cyclopentyl-3-ethyl-7-(4-methoxyphenyl)-5,6-dihydropyrazolo[3,4-e][1,4]diazepin-4(1H)-one;
3-Ethyl-1-(4-fluorophenyl)-7-phenyl-5,6-dihydropyrazolo[3,4-e][1,4]diazepin-4(1H)-one;
3-Ethyl-1-(3-nitrophenyl)-7-phenyl-5,6-dihydropyrazolo[3,4-e][1,4]diazepin-4(1H)-one;
3-Ethyl-1-[4-(methylsulfonyl)phenyl]-7-phenyl-5,6-dihydropyrazolo[3,4-e][1,4]diazepin-4(1H)-one,
1-Cyclopentyl-7-(3,4-dimethoxyphenyl)-3-ethyl-5,6-dihydropyrazolo[3,4-e][1,4]diazepin-4(1H)-one;
7-(4-Bromophenyl)-1-cyclopentyl-3-ethyl-5,6-dihydropyrazolo[3,4-e][1,4]diazepin-4(1H)-one;
1-Cyclopentyl-3-ethyl-7-(4-methylphenyl)-5,6-dihydropyrazolo[3,4-e][1,4]diazepin-4(1H)-one;
1-Cyclopentyl-3-ethyl-7-(2-methoxyphenyl)-5,6-dihydropyrazolo[3,4-e][1,4]diazepin-4(1H)-one;
Methyl 5-(1-cyclopentyl-3-ethyl-4-oxo-1,4,5,6-tetrahydropyrazolo[3,4-e][1,4]diazepin-7-yl)-2-hydroxybenzoate;
7-(4-Chlorophenyl)-1-cyclopentyl-3-ethyl-5,6-dihydropyrazolo[3,4-e][1,4]diazepin-4(1H)-one;
7-(4-Chlorophenyl)-1-cyclopentyl-3-ethyl-5,6-dihydropyrazolo[3,4-e][1,4]diazepin-4(1H)-one;
1-Cyclopentyl-7-(2,5-dimethoxyphenyl)-3-ethyl-5,6-dihydropyrazolo[3,4-e][1,4]diazepin-4(1H)-one;
7-[4-(Benzyloxy)phenyl]-1-cyclopentyl-3-ethyl-5,6-dihydropyrazolo[3,4-e][1,4]diazepin-4(1H)-one;
1-Cyclopentyl-7-(3,4-dihydro-2H-1,5-benzodioxepin-7-yl)-3-ethyl-5,6-dihydropyrazolo[3,4-e][1,4]diazepin-4(1H)-one;
1-Cyclopentyl-3-ethyl-7-(2-furyl)-5,6-dihydropyrazolo[3,4-e][1,4]diazepin-4(1H)-one;
7-tert-Butyl-1-cyclopentyl-3-ethyl-5,6-dihydropyrazolo[3,4-e][1,4]diazepin-4(1H)-one;
1-Cyclopentyl-3-ethyl-7-(3-methoxyphenyl)-5,6-dihydropyrazolo[3,4-e][1,4]diazepin-4(1H)-one;
Methyl 3-(1-cyclopentyl-3-ethyl-4-oxo-1,4,5,6-tetrahydropyrazolo[3,4-e][1,4]diazepin-7-yl)propanoate;
1-Cyclopentyl-3-ethyl-7-(4-fluorophenyl)-5,6-dihydropyrazolo[3,4-e][1,4]diazepin-4(1H)-one;
1-Cyclopentyl-3-ethyl-7-[4-(trifluoromethoxy)phenyl]-5,6-dihydropyrazolo[3,4-e][1,4]diazepin-4(1H)-one;
4-(1-Cyclopentyl-3-ethyl-4-oxo-1,4,5,6-tetrahydropyrazolo[3,4-e][1,4]diazepin-7-yl)benzonitrile;
1-Cyclopentyl-3-ethyl-7-[4-(methylsulfonyl)phenyl]-5,6-dihydropyrazolo[3,4-e][1,4]diazepin-4(1H)-one;
1-Cyclopentyl-3-ethyl-7-methyl-6-phenyl-5,6-dihydropyrazolo[3,4-e][1,4]diazepin-4(1H)-one;
1-Cyclopentyl-3-ethyl-5-methyl-7-phenyl-5,6-dihydropyrazolo[3,4-e][1,4]diazepin-4(1H)-one;
5-(1-Cyclopentyl-3-ethyl-4-oxo-1,4,5,6-tetrahydropyrazolo[3,4-e][1,4]diazepin-7-yl)-2-hydroxybenzoic acid;
3-(1-Cyclopentyl-3-ethyl-4-oxo-1,4,5,6-tetrahydropyrazolo[3,4-e][1,4]diazepin-7-yl)propanoic acid;
3-(1-Cyclopentyl-3-ethyl-4-oxo-1,4,5,6-tetrahydropyrazolo[3,4-e][1,4]diazepin-7-yl)-N-[2-(dimethylamino)ethyl]propanamide;
1-Cyclopentyl-3-ethyl-7-phenyl-5,6,7,8-tetrahydropyrazolo[3,4-e][1,4]diazepin-4(1H)-one;
3-(Methylthio)-1,7-diphenyl-5,6-dihydropyrazolo[3,4-e][1,4]diazepin-4(1H)-one;
1-(1,1-Dioxidotetrahydrothien-3-yl)-3-ethyl-7-phenyl-5,6-dihydropyrazolo[3,4-e][1,4]diazepin-4(1H)-one;
3-Ethyl-7-phenyl-1-tetrahydro-2H-thiopyran-4-yl-5,6-dihydropyrazolo[3,4-e][1,4]diazepin-4(1H)-one;
3-Ethyl-1-(1-oxidotetrahydro-2H-thiopyran-4-yl)-7-phenyl-5,6-dihydropyrazolo[3,4-e][1,4]diazepin-4(1H)-one;
1-(1,1-Dioxidotetrahydro-2H-thiopyran-4-yl)-3-ethyl-7-phenyl-5,6-dihydropyrazolo[3,4-e][1,4]diazepin-4(1H)-one;
1-Cyclopentyl-3-methyl-7-phenyl-5,6-dihydropyrazolo[3,4-e][1,4]diazepin-4(1H)-one; or,
3-Ethyl-1,7-diphenyl-5,6-dihydropyrazolo[3,4-e][1,4]diazepin-4(1H)-one;
or a pharmaceutically acceptable salt thereof.

8. A process for preparing a compound of formula (I) as claimed in claim 1, the process comprising reacting a compound of formula (II): 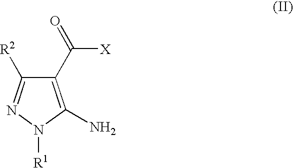
wherein X is a Cl or OH and R¹ and R² are as defined in claim 1, with an amine of formula (III): 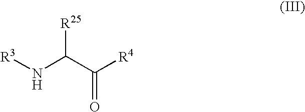
wherein R³, R⁴ and R²⁵ are as defined in claim 1, the process being carried out at a suitable temperature, in a suitable solvent and in the presence of a suitable base; and when X is OH, the process is carried out in the presence a suitable coupling agent.

9. A pharmaceutical composition which comprises a compound of the formula (I), or a pharmaceutically acceptable salt thereof as claimed in claim 1, and a pharmaceutically acceptable adjuvant, diluent or carrier.

10-11. (canceled)

12. A method of treating a PDE 4 mediated disease state in a mammal suffering from, or at risk of, said disease, which comprises administering to a mammal in need of such treatment a therapeutically effective amount of a compound of formula (I), or a pharmaceutically acceptable salt thereof as claimed in claim 1.

13. A combination comprising a compound of formula (J), or a pharmaceutically acceptable salt thereof, and one or more active agents selected from the list comprising:
a β2 adrenoceptor agonist;
a glucocorticoid receptor agonist;
a muscarinic receptor antagonist;
a modulator of chemokine receptor function; or,
an inhibitor of p38 kinase function.

14. A process for the preparation of a compound of formula (Ia) 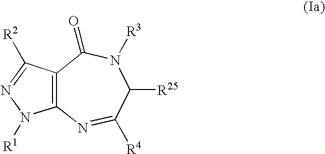
wherein:
R¹ is optionally substituted C₁₋₁₀ alkyl, or an optionally substituted 5- or 6-membered, saturated or unsaturated, carbocyclic or heterocyclic ring;
R² is optionally substituted C₁₋₁₀ alkyl, optionally substituted C₁₋₁₀ alkoxy or optionally substituted C₁₋₁₀ alkylthio;
R³ is hydrogen or optionally substituted C₁₋₁₀ alkyl;
R⁴ is optionally substituted C₁₋₁₀ alkyl, optionally substituted aryl or optionally substituted heteroaryl;
R²⁵ is hydrogen, optionally substituted aryl or optionally substituted heteroaryl;
the process comprising reacting a compound of formula (VII): 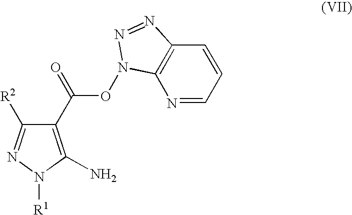
with a compound of formula (IIIa), 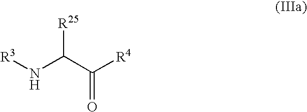
or a salt thereof, in a suitable solvent and at a suitable temperature.

15. A process as claimed in claim 14 which is carried out in the presence of a suitable base.

16. A process as claimed in claim 14 which is carried out in the presence of microwaves.
